SEQUENCE LISTING (1 sequence)

SEQ ID NO: 1 - DNA, 10 bp, synthetic construct
  source 1..10: mol_type = other DNA
aaaatttggt

(19) United States
(12) Patent Application Publication
(10) Pub. No.: US 2023/0131285 A1
(43) Pub. Date: Apr. 27, 2023

(54) IMMUNE REPERTOIRE BIOMARKERS FOR PREDICTION OF TREATMENT RESPONSE IN AUTOIMMUNE DISEASE

(71) Applicant: Life Technologies Corporation, Carlsbad, CA (US)

(72) Inventors: Shrutii SARDA, Pacifica, CA (US); Geoffrey LOWMAN, Carlsbad, CA (US)

(21) Appl. No.: 17/889,654

(22) Filed: Aug. 17, 2022

Related U.S. Application Data
(60) Provisional application No. 63/260,373, filed on Aug. 18, 2021.

(51) Int. Cl. C12Q 1/6883

(52) U.S. Cl. CPC C12Q 1/6883; C12Q 2600/158; C12Q 2600/156; C12Q 2600/106

(57) ABSTRACT

Prediction of a clinical response to a therapy of a subject with an autoimmune disease based on B cell immune repertoire may include determining a plurality of clone frequencies in a biological sample from the subject, wherein the clone frequencies include: IgM, IgD, IgG3, IgG4, IgA, IgM with first somatic hypermutation (SHM) level, IgG1 with second SHM level. A plurality of decision criteria may be applied to features including the plurality of clone frequencies. Each decision criterion applies at least one threshold to a feature, wherein the plurality of decision criteria provides a plurality of output values. The plurality of output values may be summed and a sigmoid transformation may be applied to the summed value to form a prediction value. The prediction value may be compared to a final threshold to identify the subject as a likely responder or non-responder to an autoimmune disease therapy.

### CROSS-REFERENCE TO RELATED APPLICATIONS

This application claims benefit under 35 U.S.C. § 119(e) of U.S. Provisional Application No. 63/260,373, filed Aug. 18, 2021. The entire contents of the aforementioned application are incorporated by reference herein.

### SEQUENCE LISTING

The instant application contains a Sequence Listing which has been submitted electronically in XML format and is hereby incorporated by reference in its entirety. Said XML copy, created on Dec. 23, 2022, is named TP109195USUTL1_SL.xml and is 1,961 bytes in size.

### FIELD OF THE INVENTION

The present invention relates to methods of analyzing immune repertoire biomarkers in autoimmune disease to predict treatment response using next generation sequencing (NGS), in particular methods of analyzing B cell repertoire for prediction of responders/non-responders to methotrexate (MTX) treatment for rheumatoid arthritis.

### BRIEF DESCRIPTION OF THE DRAWINGS

FIG. 1 shows an example of a plot of drops in feature accuracy for the leave-one-out strategy applied to the features.

FIGS. 2A to 2X show diagrams of a plurality component decision trees of a decision tree structure in accordance with an exemplary embodiment.

FIG. 3A shows an example of the confusion matrix for the prediction of responders (R) and non-responders (N).

FIG. 3B shows an example of the ROC curve (receiver operating characteristic curve) of the final model on the dataset.

FIG. 4 shows an example of a plot of the relative importance of the features in the final prediction.

FIG. 5 shows an example of a waterfall chart illustrating the relative impact of the features on the probability of response for observation #39 (subject #39).

FIG. 6 shows an example of a waterfall chart illustrating the relative impact of the features on the probability of response for observation #52 (subject #52).

FIG. 7 shows an example of a set of sixteen features in order of importance.

FIG. 8A shows an example of the confusion matrix for the prediction of responders (R) and non-responders (N).

FIG. 8B shows an example of the ROC curve of the final model on the dataset.

FIG. 9 is a diagram of an exemplary workflow for removal of PCR or sequencing-derived errors using stepwise clustering of similar CDR3 nucleotides sequences with steps: (A) very fast heuristic clustering into groups based on similarity (cd-hit-est); (B) cluster representative chosen as most common sequence, randomly picked for ties; (C) merge reads into representatives; (D) compare representatives and if within allotted hamming distance, merge clusters.

FIG. 10 is a diagram of an exemplary workflow for removal of residual insertion/deletion (indel) error by comparing homopolymer collapsed CDR3 sequences using Levenshtein distance with the steps: (A) collapse homopolymers and calculate Levenshtein distances between cluster representatives; (B) merge reads that now cluster together, these represent complex indel errors; (C) report lineages to user.

### DETAILED DESCRIPTION

Methotrexate is commonly employed as a first line treatment for rheumatoid arthritis, although only a subset of recipients experiences a durable remission of disease symptoms. Those who do not respond favorably have the option of receiving alternative therapies. Owing to the slow kinetics of methotrexate activity, where the first signs of disease relief are typically observed 4-6 weeks post initiation of therapy, evaluating response requires extended monitoring during which time an individual may continue to suffer symptoms of disease. Furthermore, a subset of individuals may show temporary remission of symptoms for several months duration before ultimately returning to a pretreatment diseased state. Therefore, there is an outstanding need for biomarkers predictive of response to treatment (e.g., methotrexate). Such biomarkers would reduce the time required to identify an effective therapy, thereby improve patient outcomes and reducing cost of treatment.

In some embodiments, methods, compositions and analysis provided herein for use in methods of predicting clinical responsiveness of a subject with autoimmune disease to a therapy comprise determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SEIM) level clone frequency, an IgG1 with a second somatic hypermutation (SEIM) level clone frequency. A plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values. The output values are added to give a summed value. A sigmoid transformation is applied to the summed value to form a prediction value. The prediction value is compared to a final threshold to identify the subject as a likely responder or a likely non-responder to an autoimmune disease therapy (e.g. methotrexate).

Peripheral blood leukocytes (PBL) were extracted from 99 rheumatoid arthritis patients at baseline (time of first methotrexate administration), month 6 and month 12 post-treatment. A sample of 25 ng. of total RNA from PBL was used for targeted AMPLISEQ™ IGH sequencing via the ONCOMINE™ IGH-LR assay (Thermo Fisher Scientific), an NGS assay, with 7 samples per 530 chip on the GENESTUDIO™ S5 (Thermo Fisher Scientific), and sequenced to a target of 1.5M reads per sample. RNA was converted to cDNA by reverse transcription, followed by targeted amplification of the cDNA using the ONCOMINE™ IGH-LR assay. In other approaches, another library preparation and next generation sequencing (NGS) based assay that provides the sequence of the VDJ-C region of the IGH could be used. Reads from the sequencer were processed and analyzed via the ION REPORTER′ Software (Thermo Fisher Scientific) using the BCR IGH-LR workflow with settings for bidirectional support required and full length read required set to “true”. The BCR IGH-LR workflow provided metrics, including individual clone sequences, their frequencies, isotype, somatic hypermutation collected across the whole cohort. Sample metadata pertaining to age, gender, clinical disease activity index score (CDAI), smoking status and treatment response information (R: responder or N: non-responder) were collected from the site of collection.

Data exploration and aggregation across responders and non-responders revealed certain trends of differences in their clinical disease activity score indices, isotype specific clone frequencies, somatic hypermutation patterns, frequencies of highly mutated clones, and lineage relationships between clones of different isotypes. These patterns were complex and inter-dependent, consistent with the heterogenous pathology presented by rheumatoid arthritis (RA) patients. Features suggestive of association with response include CDAI, IGHM highly mutated (>10%) clone frequency, IGHA2 highly mutated (>10%) clone frequency, IGHG1 highly mutated (>10%) clone frequency, IGHM clone frequency, IGHD clone frequency, IGHA1+IGHA2 clone frequency, IGHG1 clone frequency, IGHG3+IGHG4 clone frequency, and IGHM average somatic hypermutation level (SHM), IGHA1+IGHA2 average somatic hypermutation level (SHM) and IGHG1 average somatic hypermutation level (SHM). As used herein, clone frequency is the number of reads identified for a particular clone divided by the total number of reads. As used herein, terms like IGHA1+IGHA2 clone frequency means the sum of the clone frequencies for IGHA1 and IGHA2. Terms like IGHA1+IGHA2 average somatic hypermutation level (SHM) means the sum of the average SHMs for IGHA1 and IGHA2. Highly mutated clone frequencies have SHM levels greater than a threshold SEM level. The SHM level threshold may be a value between about 5% to about 15% for designating highly mutated clone frequencies. In some embodiments, a preferred value of the threshold SHM level is 10%. In other examples, a threshold SHM level of 1% 2%, 3%, 4%, 5%, 6%, 7%, 8%, 9%, 10%, 11%, 12%, 13%, 14% or 15% may be used. These features were provided as feature inputs to a de novo random forest (RF) machine learning model, where the predictor was encoded as a binary variable representing responders and non-responders.

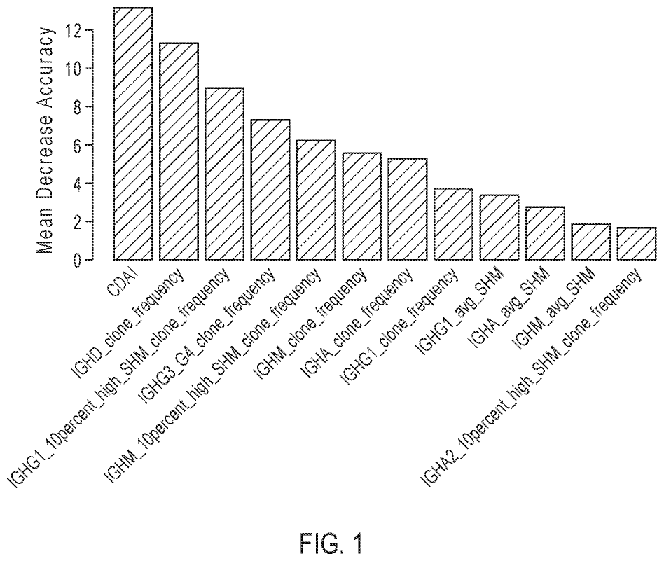

Selected features include CDAI, IGHM clone frequency, IGHD clone frequency, IGHA1+IGHA2 clone frequency, IGHG3+IGHG4 clone frequency, IGHM clone frequency and IGHG1 highly mutated (>10% SHM) clone frequency, based on having high predictive importance based on optimally parameterized and best fit RF model. These features were selected based on a feature importance curve generated by a leave-one-out strategy, showing per feature the mean decrease in accuracy of the resulting model when it was left out of the model. FIG. 1 shows an example of a plot of drops in feature accuracy for the leave-one-out strategy applied to the features. The higher the drop in accuracy, the more important a given feature was deemed, and the “elbow” of the drop was determined to be at the 7^{th} most important feature.

A plurality of decision criteria may be applied to the features to predict whether the subject is a likely responder/non-responder to treatment. The plurality of decision criteria may be organized as a plurality of component decision trees. Each component decision tree may apply at least one threshold to at least one feature to provide a corresponding output value.

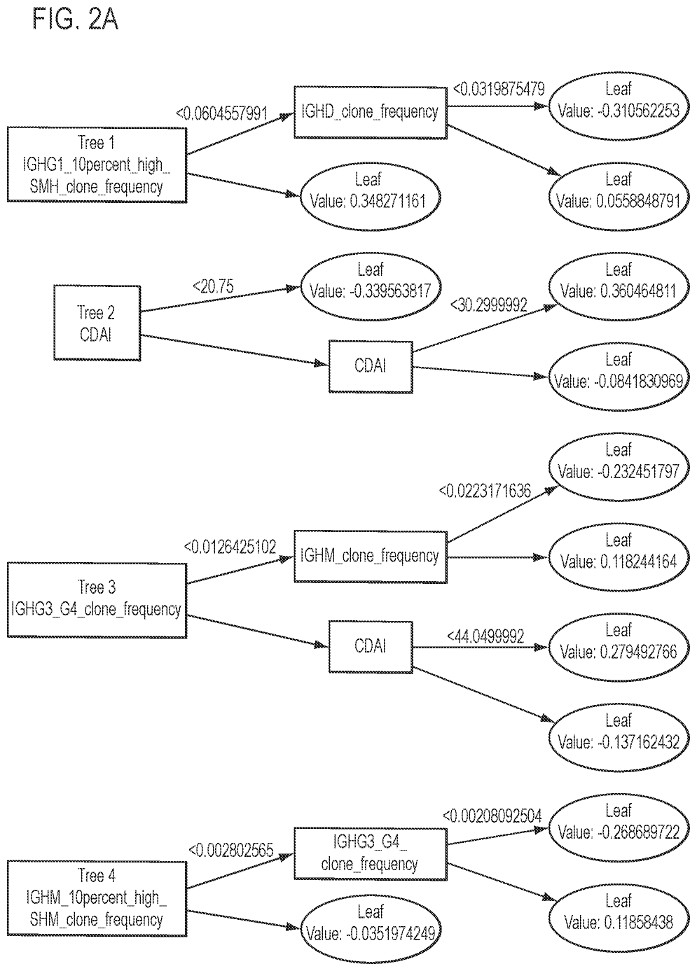

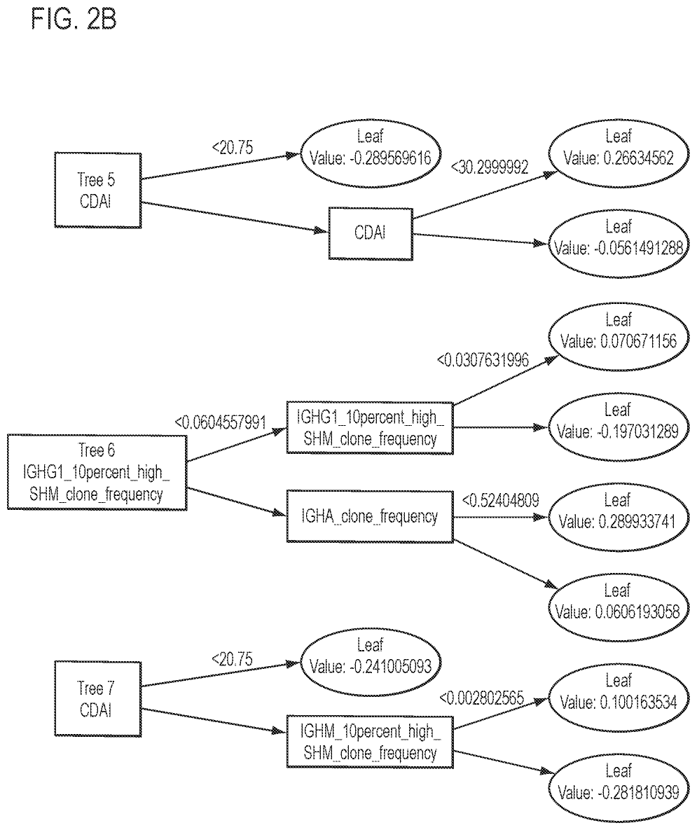

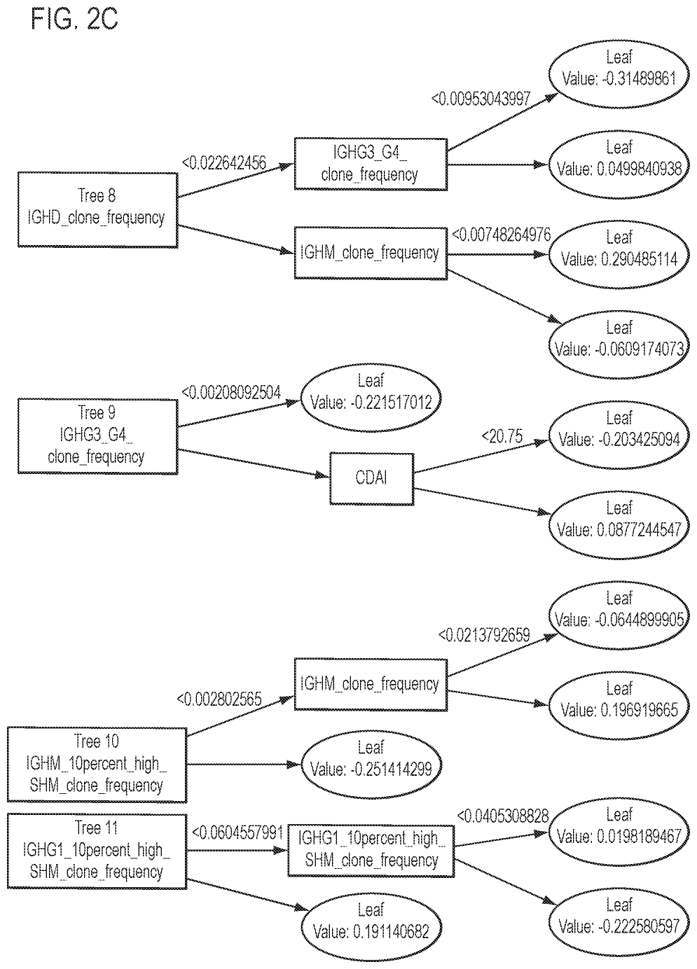

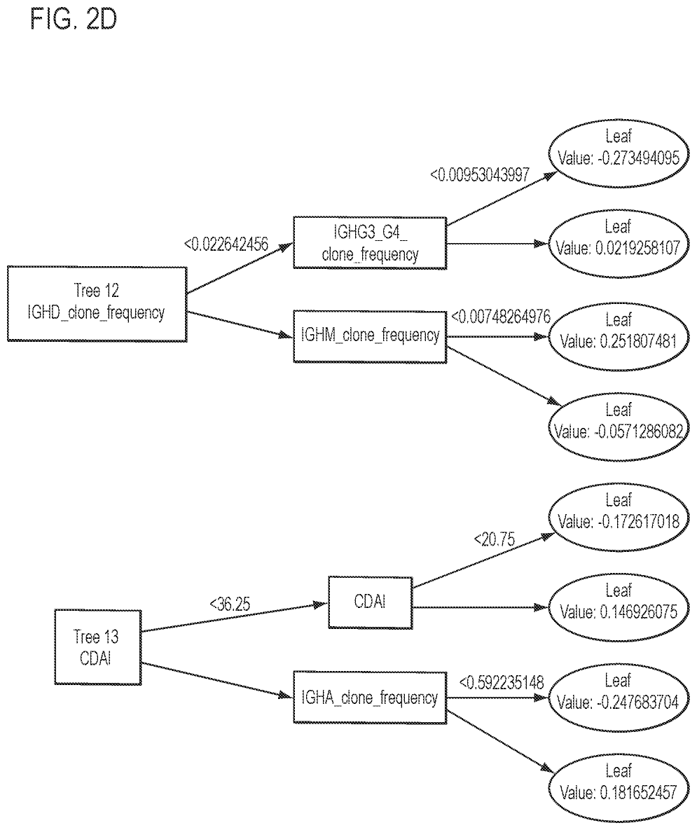

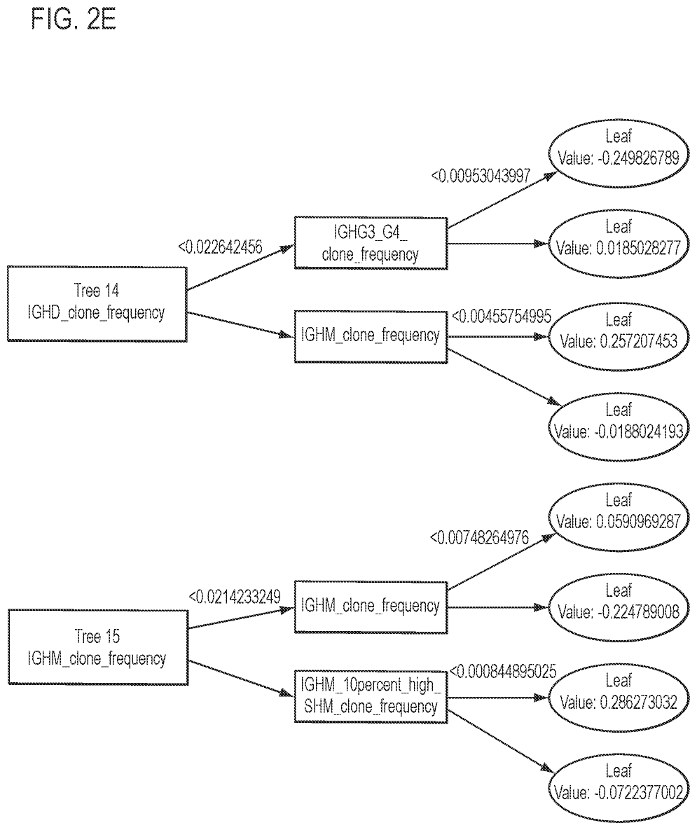

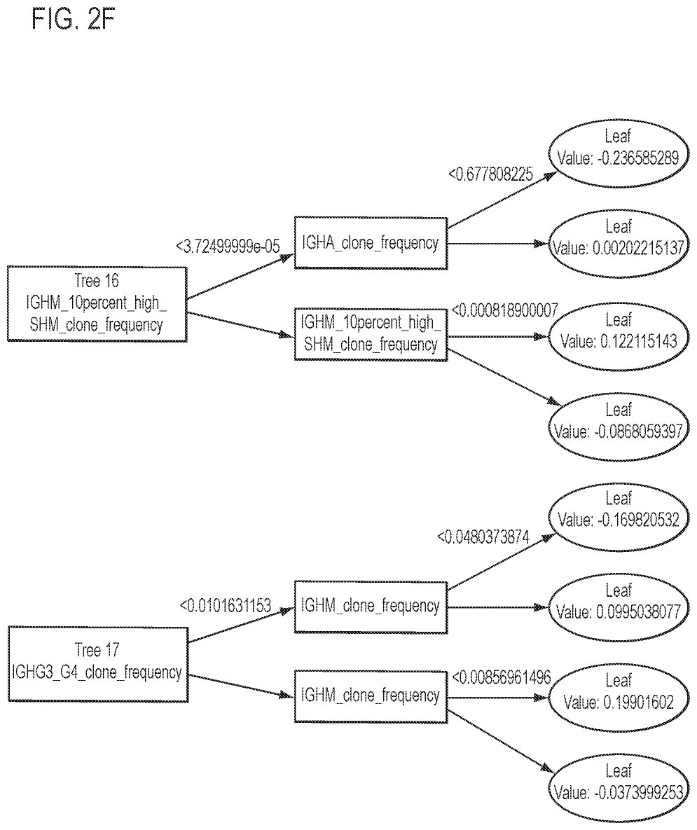

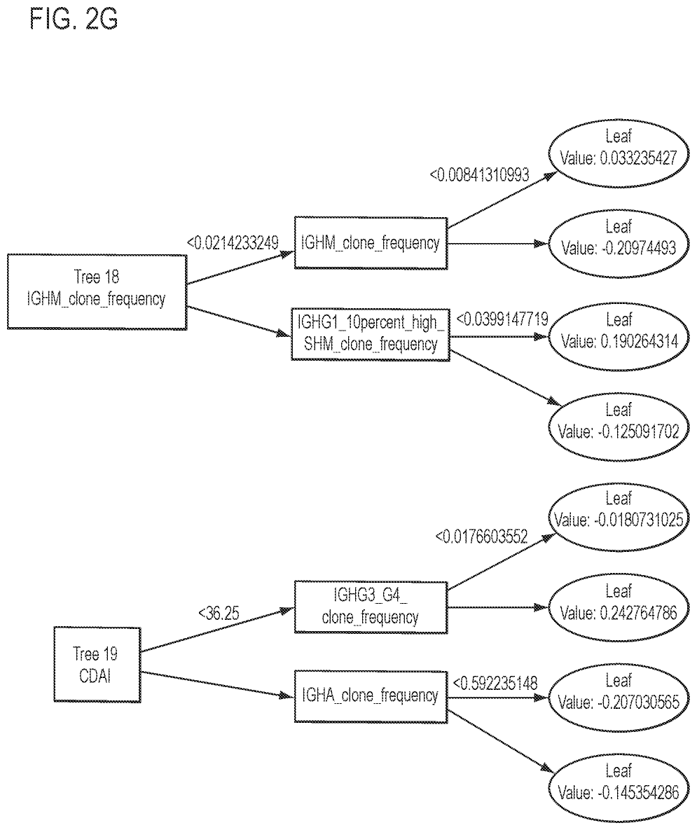

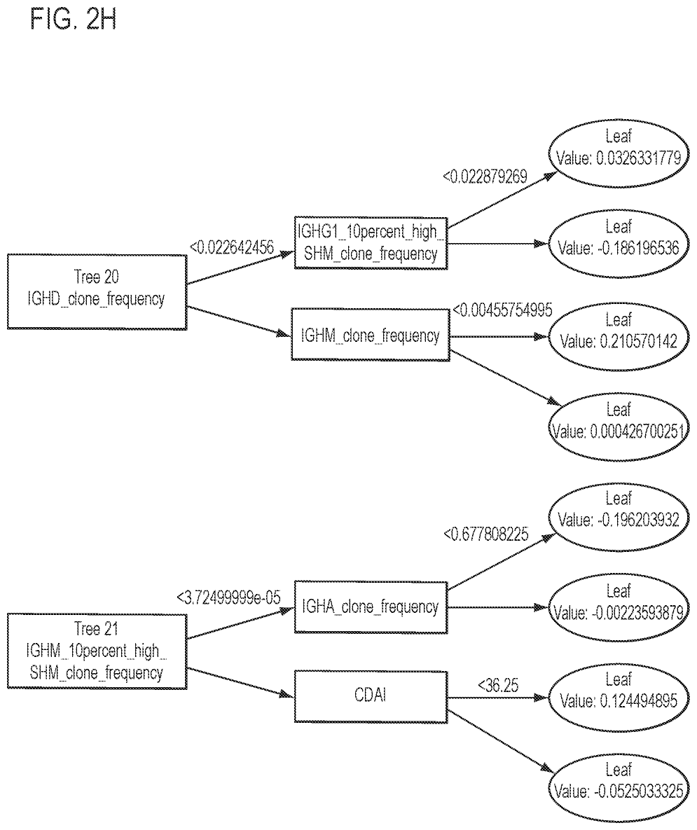

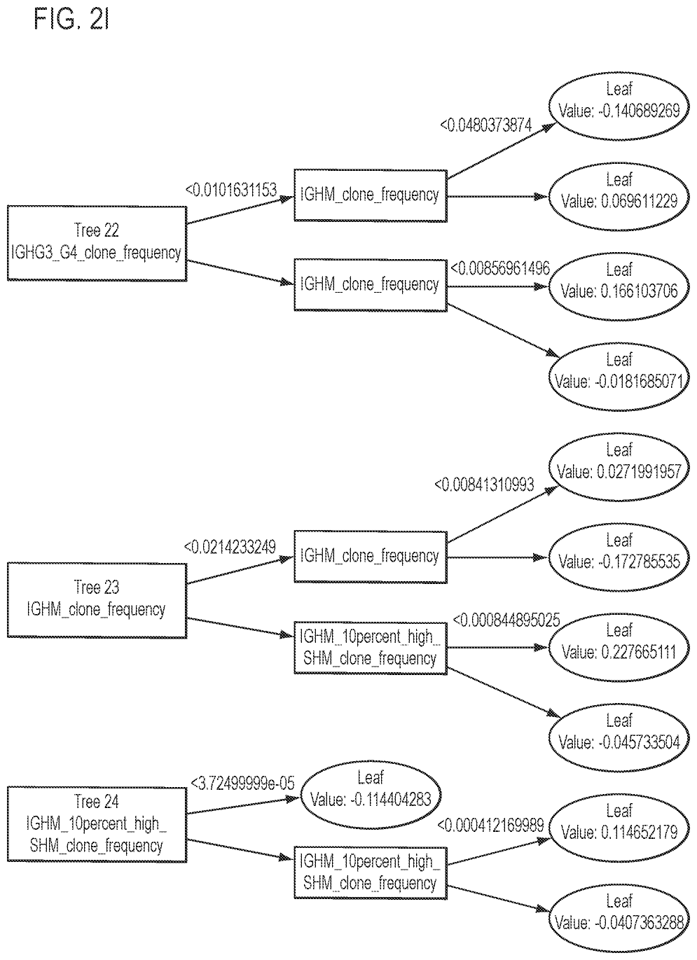

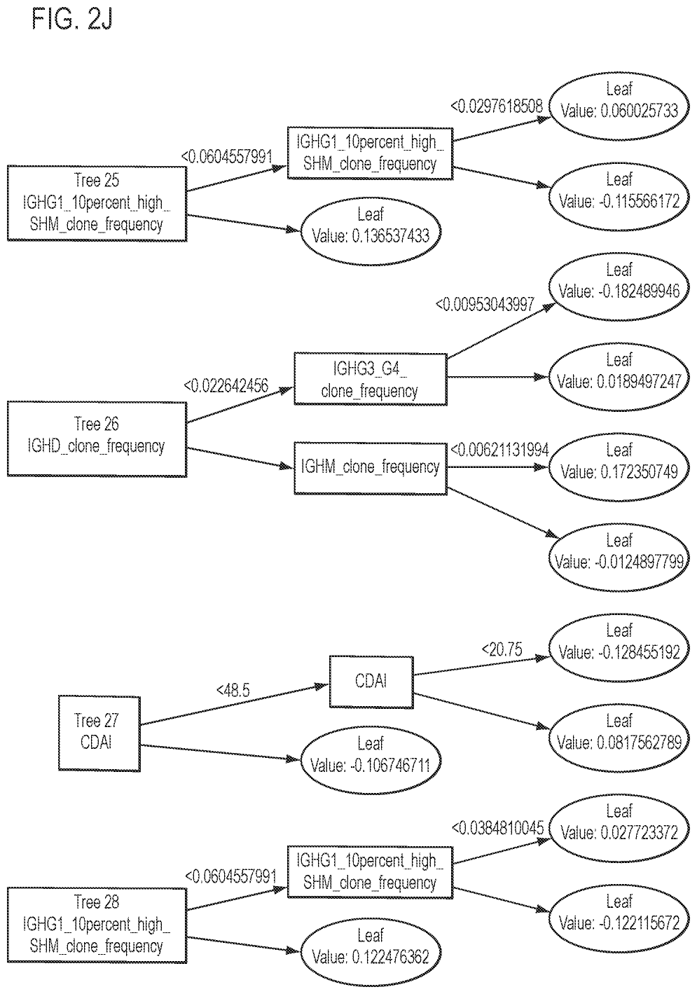

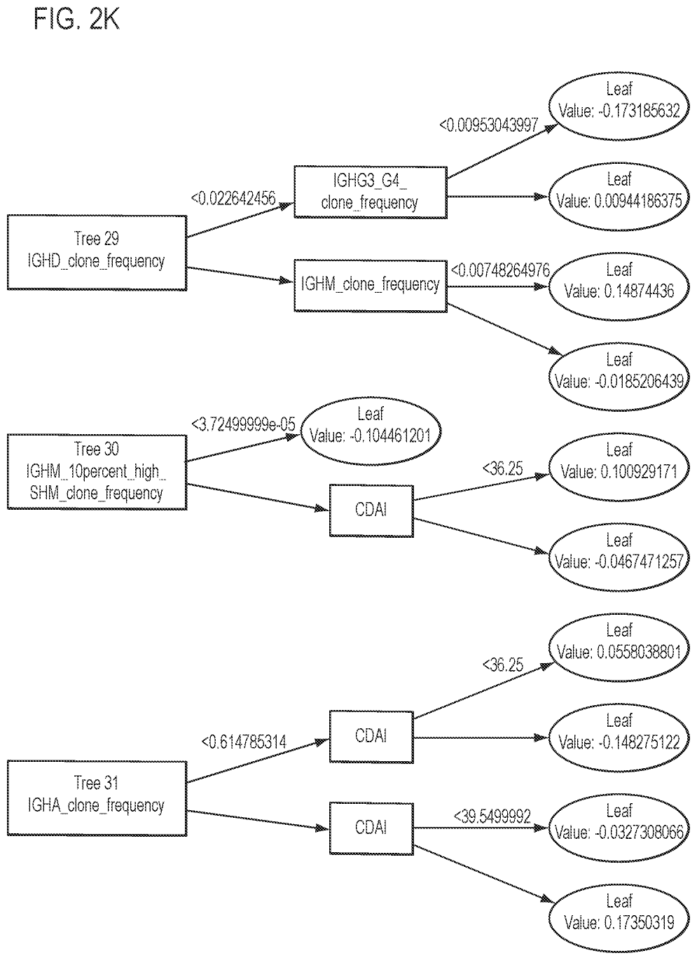

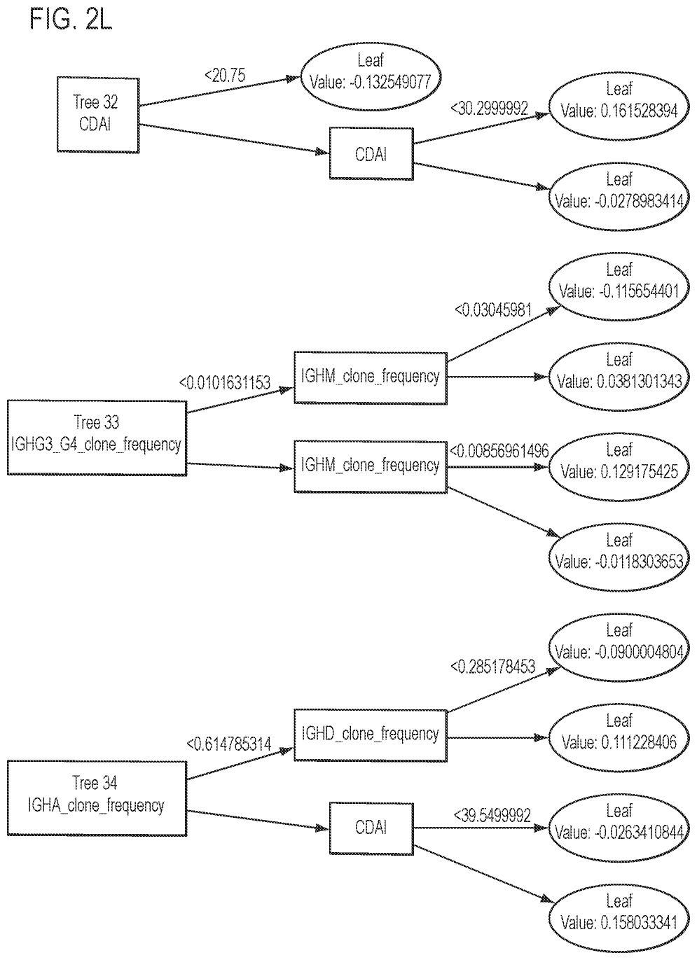

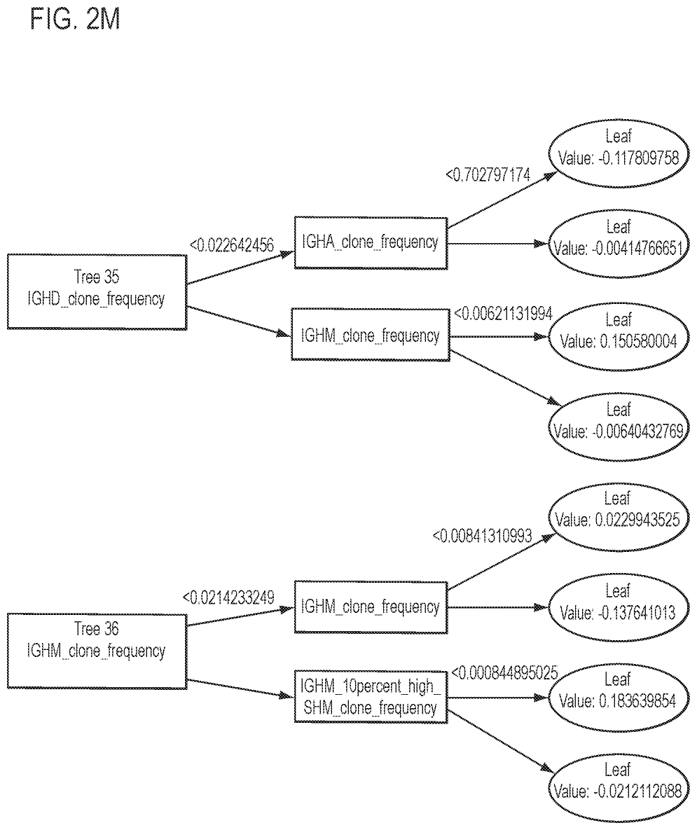

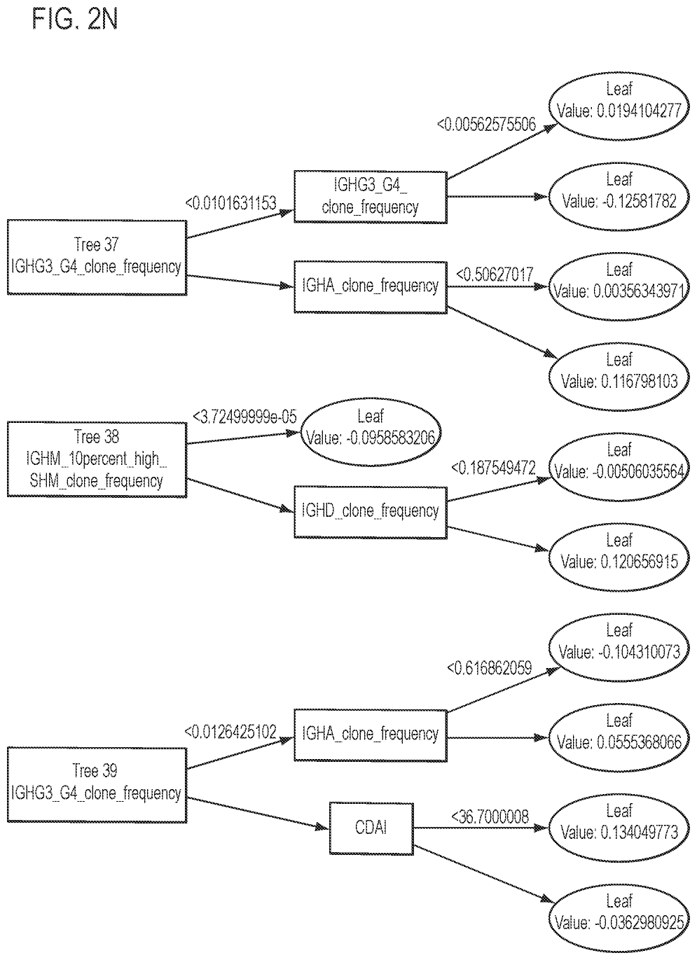

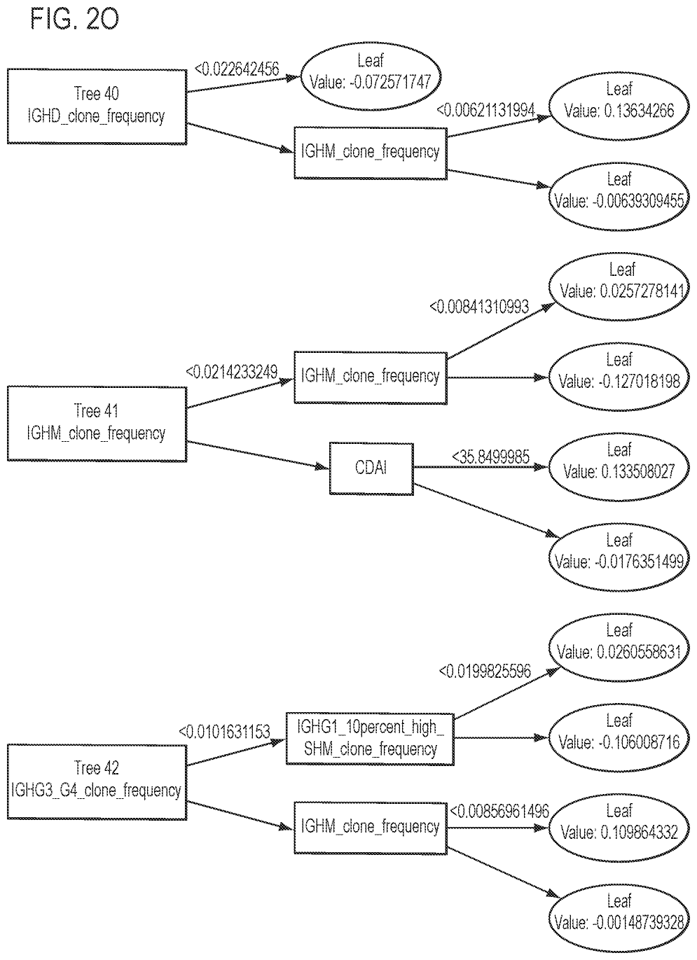

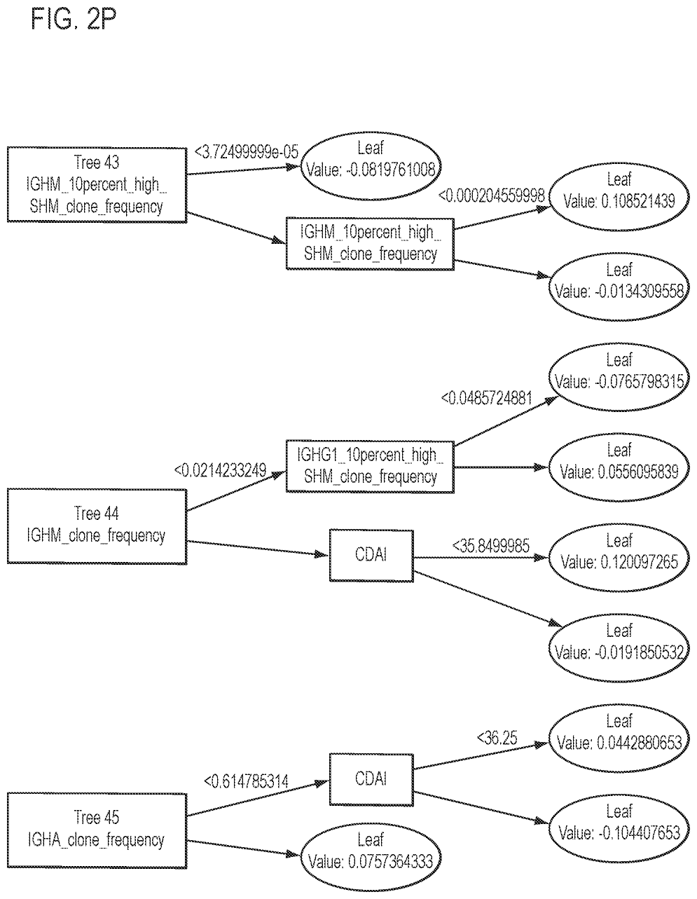

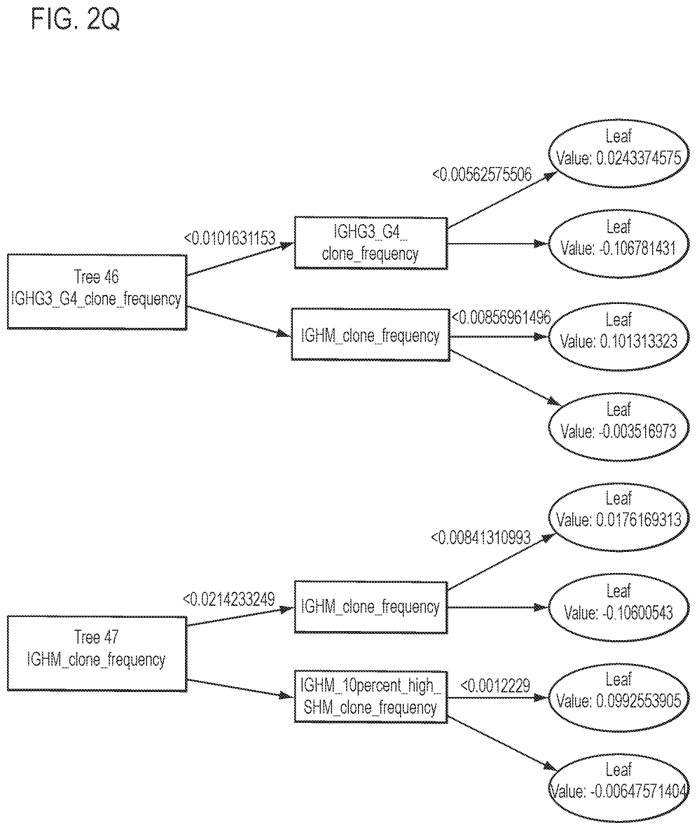

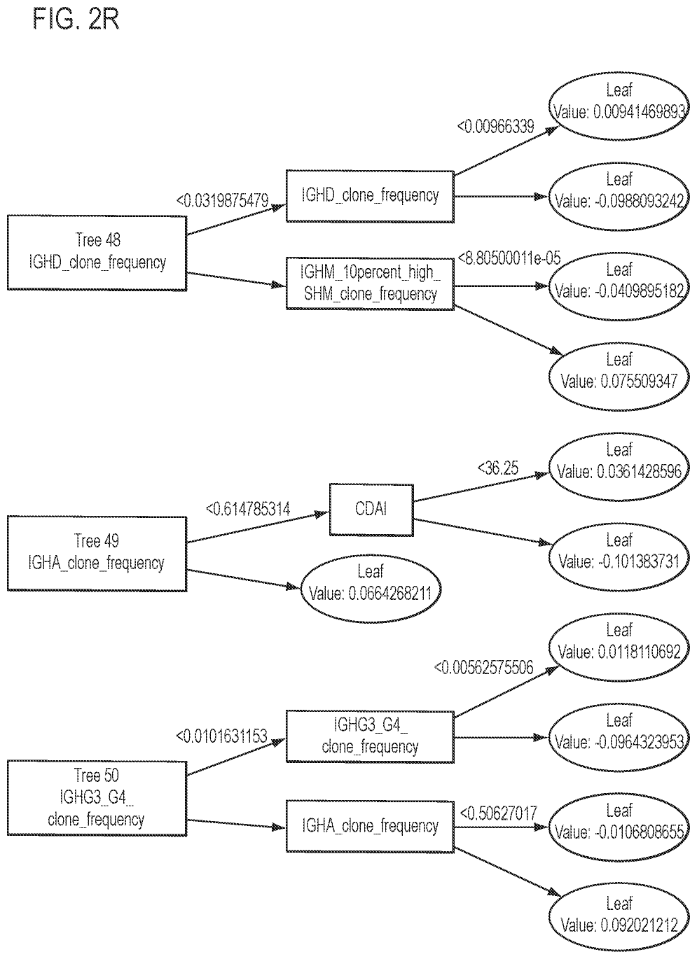

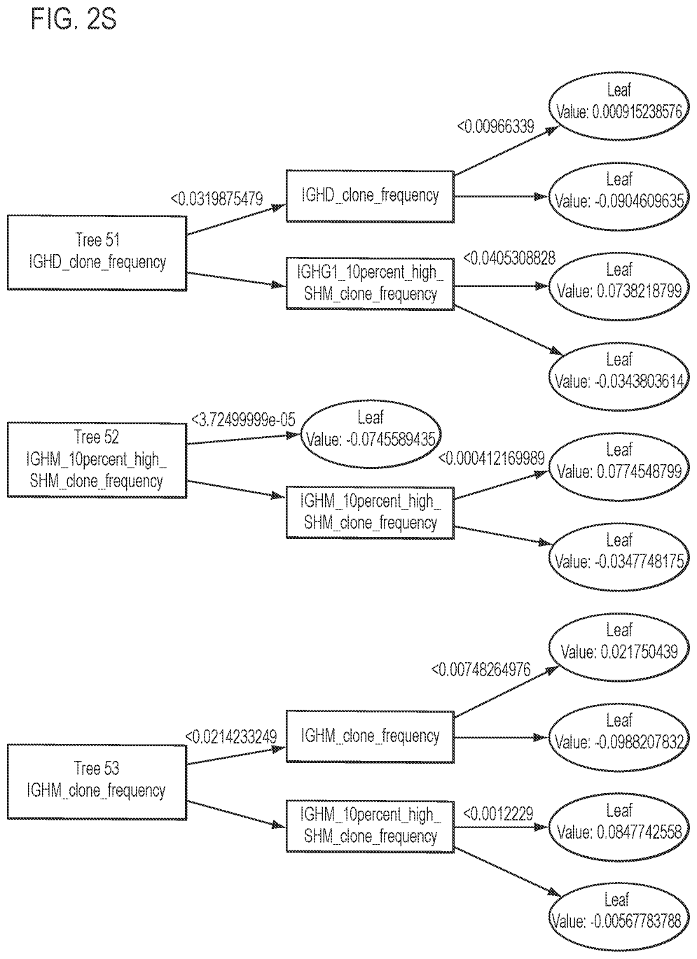

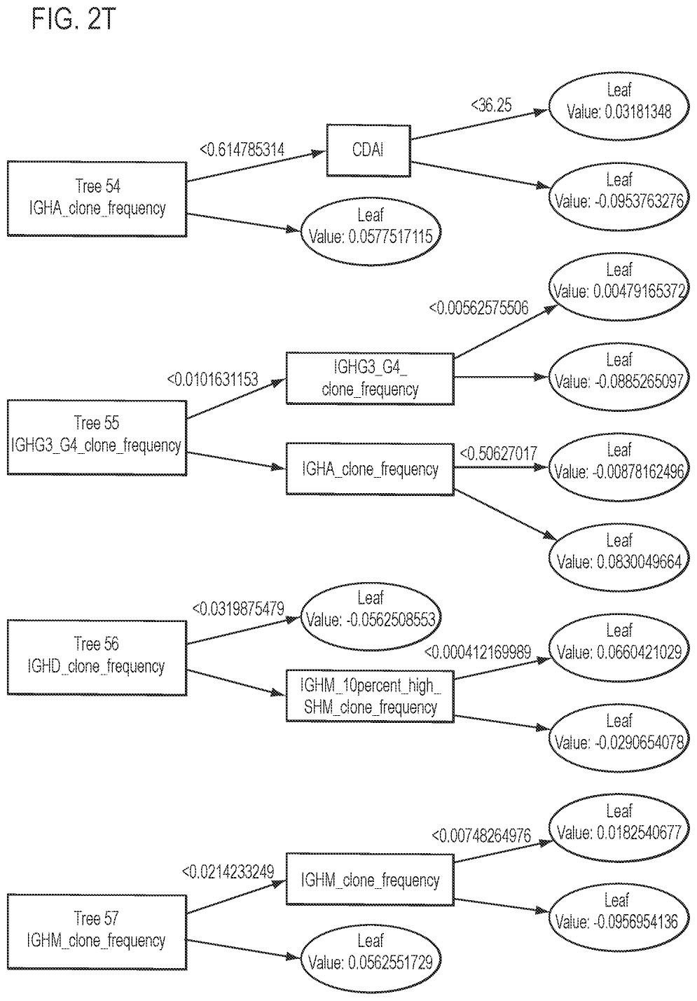

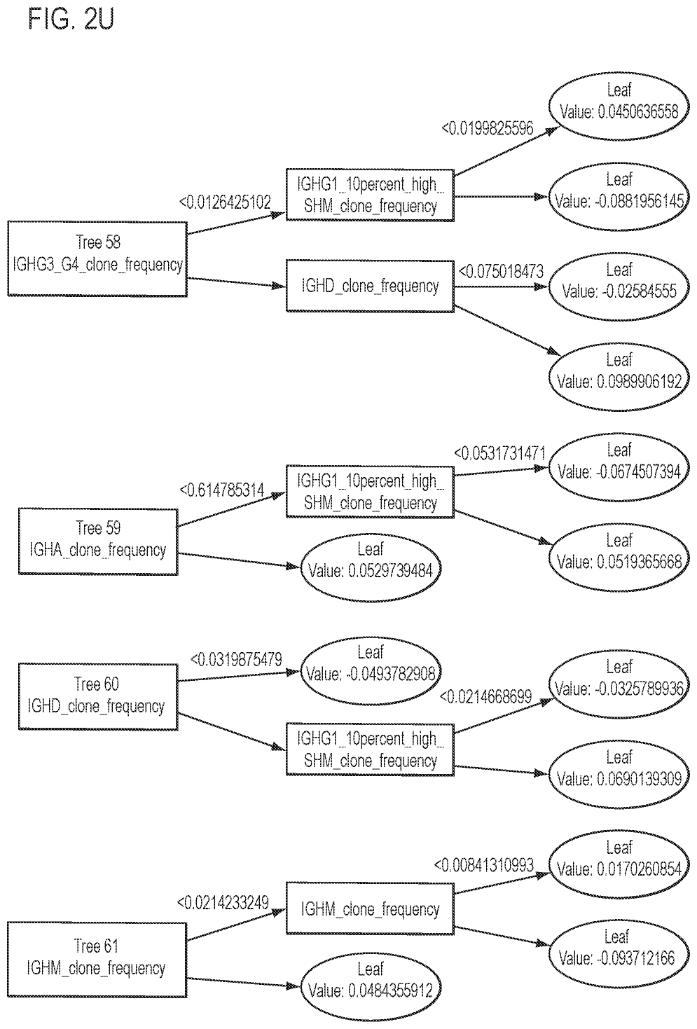

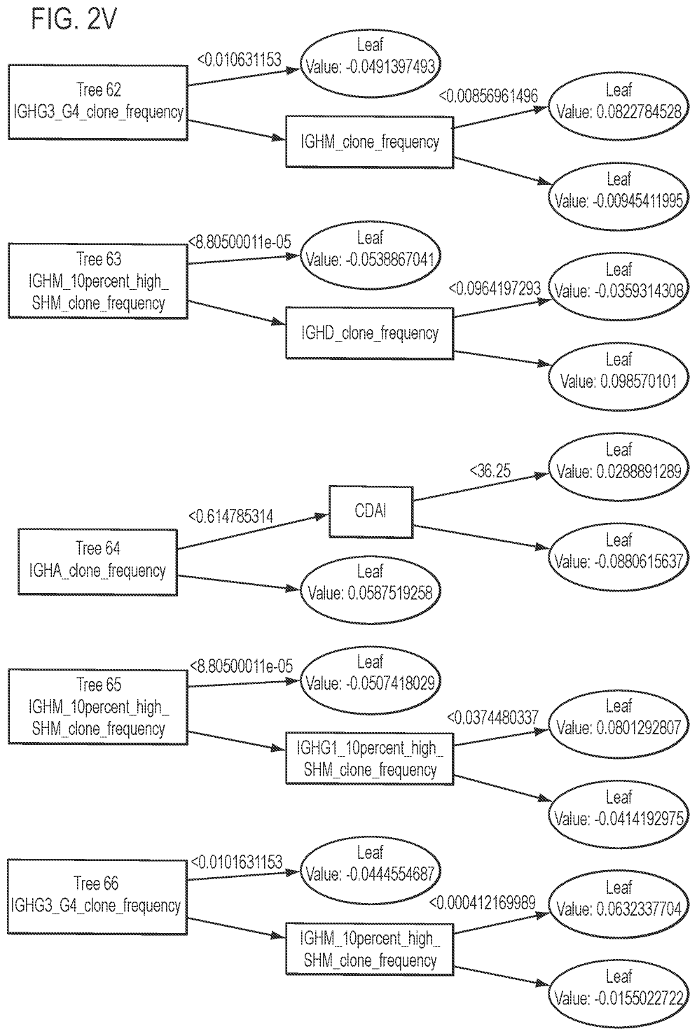

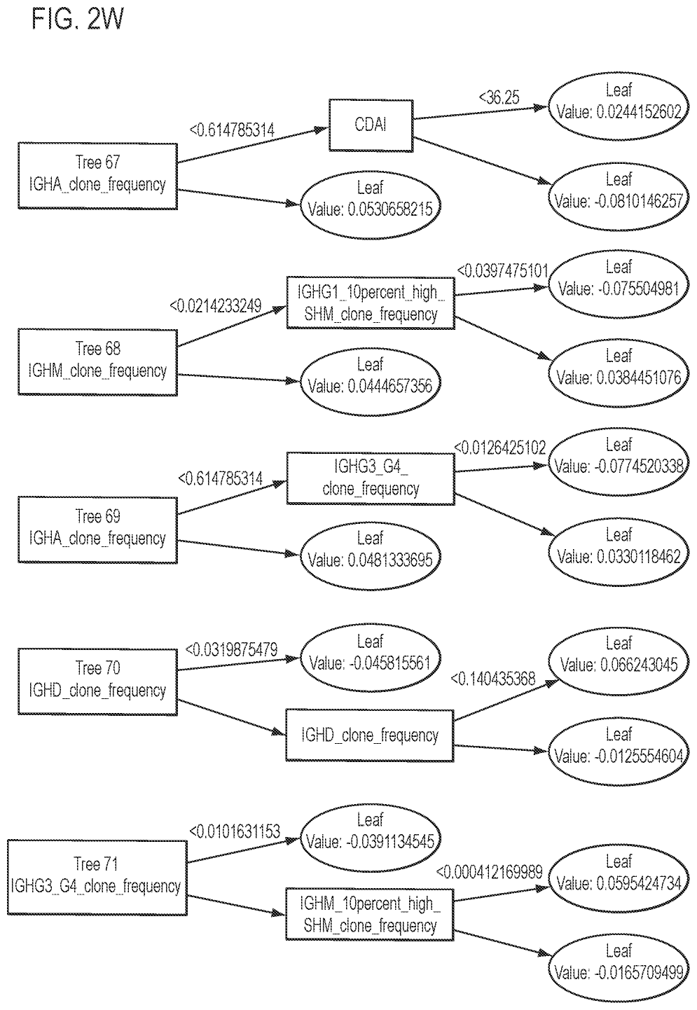

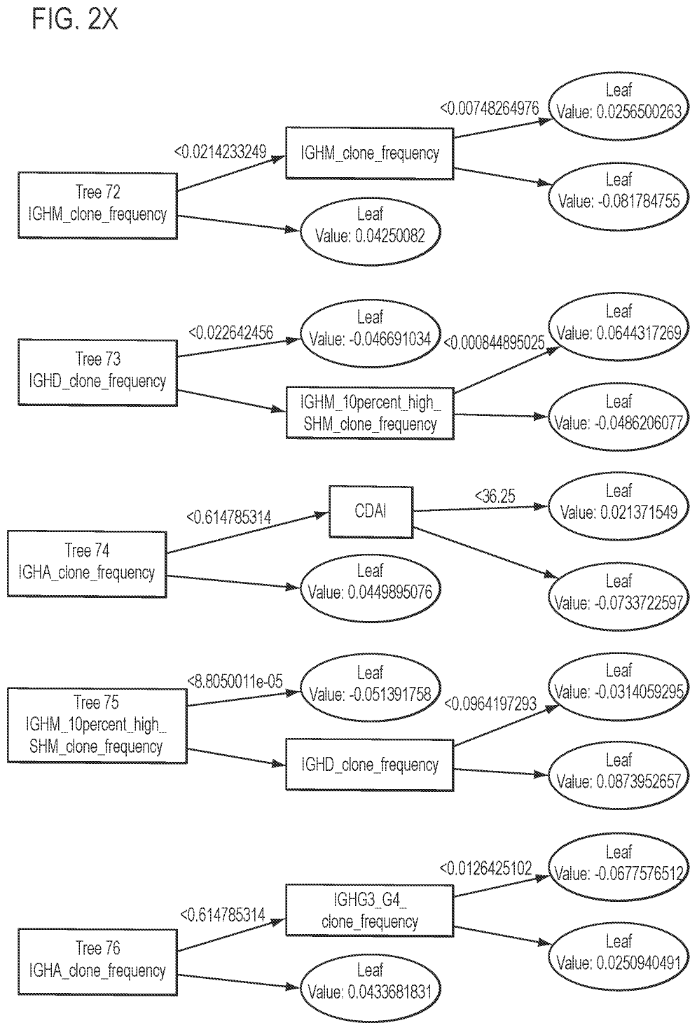

FIGS. 2A to 2X show diagrams of a plurality component decision trees of a decision tree structure in accordance with an exemplary embodiment. For each component decision tree, the blocks give the input features for the and the ellipses (“Leaf”) give the possible output values indicated by “Value”. The numbers preceded by the less than symbol (<number) give the threshold values applied to the input features. For a component decision tree having one input feature, up to two threshold values may be applied, depending on the feature value, and one of three possible output values is selected by the decision logic. For a component decision tree having two input features, up to two threshold values may be applied, depending on the features' values, and one of three possible output values is selected by the decision logic. For a component decision tree having three input features, two threshold values of three possible threshold values may be applied, depending on the features' values, and one of four possible output values is selected by the decision logic. This decision tree structure of FIGS. 2A to 2X includes 76 component decision trees.

For example, the component decision tree, Tree 1, in FIG. 2A, the input features include the IgG1 with a somatic hypermutation (SEIM) level of greater than 10% clone frequency (IGHG1_10percent_high_SEIM_clone_frequency) and the IgD clone frequency (IGHD_clone_frequency). Tree 1 may apply up to two thresholds and has three possible output values. Tree 1 applies the following logic:

```
If IGHG1_10percent_high_SHM_clone_frequency < 0.0604557991,
  If IGHD_clone_frequency < 0.0319875479,
    Value = −0.310562253
  Else
    Value = 0.0558848791
Else
  Value = 0.348271161
```

For example, the component decision tree, Tree 2, in FIG. 2A has one feature CDAI as input and may apply up to two thresholds and has three possible output values. Tree 2 applies the following logic:

```
If CDAI < 20.75,
  Value = −0.339563817
Else
  If CDAI < 30.2999992,
    Value = 0.360464811
  Else
    Value = −0.0841830969
```

For example, the component decision tree, Tree 3, in FIG. 2A, has three input features, IGHG3+IGHG4 clone frequency (IGHG3_G4_clone_frequency), IGHM clone frequency (IGHM clone frequency) and CDAI. Tree 3 may apply two of three thresholds and has four possible output values. Tree 3 applies the following logic:

```
If IGHG3_G4_clone_frequency < 0.0126425102,
  If IGHM_clone_frequency < 0.0223171636,
    Value = −0.232451797
  Else
    Value = 0.118244164
Else
  If CDAI < 44.0499992,
    Value = 0.279492766
  Else
    Value = −0.137162432
```

The plurality of decision trees applied to the input features provide a plurality of output values, one output value for each component decision tree. The sum of the plurality of output values may be calculated as SUM=v(1)+v(2)+ . . . v(N), where v(i) is the output value from the i-th decision tree and N is the number of decision trees. In some embodiments, the number of decision trees N=76.

A sigmoid function σ may be applied to the sum to form a prediction value, as follows:

Prediction value=σ(SUM) (1)

where σ(X)=1/(1+exp(−x)). The prediction value is between 0 and 1. The prediction value is compared to a final threshold to identify the subject as a likely responder or a likely non-responder. In some embodiments, the value of final threshold 0.5 is preferred. In some embodiments, range of final thresholds is between about 0.4 to about 0.57. This range of thresholds provides sensitivity and specificity between about 74% to about 84%. In certain embodiments, the final threshold is about 0.40, 0.41, 0.42, 0.43, 0.44, 0.45, 0.46, 0.47, 0.48, 0.49, 0.50, 0.51, 0.52, 0.53, 0.54, 0.55, 0.56, or 0.57.

In some embodiments, methods for use with the present teachings may include one or more features described in PCT international publication number WO 2021/151114A1, published 29 Jul. 2021, which is incorporated by reference herein in its entirety.

The phrase “next generation sequencing,” or NGS, refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

In some embodiments a multiplex next generation sequencing workflow is used for effective detection and analysis of the immune repertoire in a sample in conjunction with provided methods. Provided methods utilize workflows, compositions, systems, and kits for use in high accuracy amplification and sequencing of immune cell receptor sequences (e.g., T cell receptor (TCR), B cell receptor (BCR or Ab) targets) in monitoring and resolving complex immune cell repertoire(s) in a subject. The target immune cell receptor genes have undergone rearrangement (or recombination) of the VDJ or VJ gene segments, the gene segments depending on the particular receptor gene (e.g., IgH, IgK, TCR beta or TCR alpha). In certain embodiments, the present disclosure provides methods for use of workflows, compositions, and systems that use nucleic acid amplification, such as polymerase chain reaction (PCR), to enrich expressed variable regions of immune receptor target nucleic acid for subsequent sequencing. In certain embodiments, the present disclosure provided methods utilize workflows, compositions, and systems that use nucleic acid amplification, such as PCR, to enrich rearranged target immune cell receptor gene sequences from gDNA for subsequent sequencing. In certain embodiments, the present disclosure also provides methods for use of workflows and systems for effective identification and removal of amplification or sequencing-derived error(s) to improve read assignment accuracy and lower the false positive rate. In particular, provided methods described herein may improve accuracy and performance in sequencing applications with nucleotide sequences associated with genomic recombination and high variability. In some embodiments, methods, compositions, systems, and kits provided herein are for use in amplification and sequencing of the complementarity determining regions (CDRs) of an expressed immune receptor in a sample. In some embodiments, methods, compositions, systems, and kits provided herein are for use in amplification and sequencing of the CDRs of rearranged immune cell receptor gDNA in a sample. Thus, multiplex immune cell receptor expression compositions and immune cell receptor gene-directed compositions for multiplex library preparation, used in conjunction with next generation sequencing technologies and workflow solutions (e.g., manual or automated), can be used for effective detection and characterization of the immune repertoire in a sample in conjunction with the methods provided herein.

The CDRs of a TCR or BCR result from genomic DNA undergoing recombination of the V(D)J gene segments as well as addition and/or deletion of nucleotides at the gene segment junctions. Recombination of the V(D)J gene segments and subsequent hypermutation events leads to extensive diversity of the expressed immune cell receptors. With the stochastic nature of V(D)J recombination, it is often the case that rearrangement of the T or B cell receptor genomic DNA will fail to produce a functional receptor, instead producing what is termed an “unproductive” rearrangement. Typically, unproductive rearrangements have out-of-frame Variable and Joining coding segments, and lead to the presence of premature stop codons and synthesis of irrelevant peptides. Unproductive TCR or BCR gene rearrangements are generally rare in cDNA-based repertoire sequencing for a number of biological or physiological reasons such as: 1) nonsense-mediated decay, which destroys mRNA containing premature stop codons, 2) B and T cell selection, where only B and T cells with a functional receptor survive, and 3) allelic exclusion, where only a single rearranged receptor allele is expressed in any given B or T cell.

Accordingly, in some embodiments, methods and compositions provided herein are used for amplifying the recombined, expressed variable regions of immune cell receptor mRNA, e.g. BCR and/or TCR mRNA. In some embodiments, RNA extracted from biological samples is converted to cDNA. Multiplex amplification is used to enrich for a portion of BCR or TCR cDNA which includes at least a portion of the variable region of the receptor. In some embodiments, the amplified cDNA includes one or more complementarity determining regions CDR1, CDR2, and/or CDR3 for the target receptor. In some embodiments, the amplified cDNA includes one or more complementarity determining regions CDR1, CDR2, and/or CDR3 for immunoglobulin heavy chain (IgH).

BCR and TCR sequences can also appear as unproductive rearrangements from errors introduced during amplification reactions or during sequencing processes. For example, an insertion or deletion (indel) error during a target amplification or sequencing reaction can cause a frameshift in the reading frame of the resulting coding sequence. Such a change may result in a target sequence read of a productive rearrangement being interpreted as an unproductive rearrangement and discarded from the group of identified clonotypes. Accordingly, in some embodiments, methods and systems provided herein include processes for identification and/or removing PCR or sequencing-derived error from the determined immune receptor sequence.

In some embodiments, methods and compositions provided are used for amplifying the rearranged variable regions of immune cell receptor gDNA, e.g., rearranged BCR and/or TCR gene DNA. Multiplex amplification is used to enrich for a portion of rearranged BCR or TCR gDNA which includes at least a portion of the variable region of the receptor. In some embodiments, the amplified gDNA includes one or more complementarity determining regions CDR1, CDR2, and/or CDR3 for the target receptor. In some embodiments, the amplified gDNA includes one or more complementarity determining regions CDR1, CDR2, and/or CDR3 for IgH. In some embodiments, the amplified gDNA includes primarily CDR3 for the target receptor, e.g., CDR3 for IgH.

As used herein, “immune cell receptor” and “immune receptor” are used interchangeably.

As used herein, the terms “complementarity determining region” and “CDR” refer to regions of a T cell receptor or an antibody (immunoglobulin) where the molecule complements an antigen's conformation, thereby determining the molecule's specificity and contact with a specific antigen. In the variable regions of T cell receptors and antibodies, the CDRs are interspersed with regions that are more conserved, termed framework regions (FR). Each variable region of a T cell receptor and an antibody contains 3 CDRs, designated CDR1, CDR2 and CDR3, and also contains 4 framework sub-regions, designated FR1, FR2, FR3 and FR4.

As used herein, the term “framework” or “framework region” or “FR” refers to the residues of the variable region other than the CDR residues as defined herein. There are four separate framework sub-regions that make up the framework: FR1, FR2, FR3, and FR4.

The particular designation in the art for the exact location of the CDRs and FRs within the receptor molecule (TCR or immunoglobulin) varies depending on what definition is employed. Unless specifically stated otherwise, the IMGT designations are used herein in describing the CDR and FR regions (see Brochet et al. (2008) Nucleic Acids Res. 36:W503-508, herein specifically incorporated by reference). As one example of CDR/FR amino acid designations, the residues that make up the FRs and CDRs of T cell receptor beta have been characterized by IMGT as follows: residues 1-26 (FR1), 27-38 (CDR1), 39-55 (FR2), 56-65 (CDR2), 66-104 (FR3), 105-117 (CDR3), and 118-128 (FR4).

Other well-known standard designations for describing the regions include those found in Kabat et al., (1991) Sequences of Proteins of Immunological Interest, 5th Ed. Public Health Service, National Institutes of Health, Bethesda, Md., and in Chothia and Lesk (1987) J. Mol. Biol. 196:901-917; herein specifically incorporated by reference. As one example of CDR designations, the residues that make up the six immunoglobulin CDRs have been characterized by Kabat as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; and by Chothia as follows: residues 26-32 (CDRL1), 50-52 (CDRL2) and 91-96 (CDRL3) in the light chain variable region and 26-32 (CDRH1), 53-55 (CDRH2) and 96-101 (CDRH3) in the heavy chain variable region.

The term “T cell receptor” or “T cell antigen receptor” or “TCR,” as used herein, refers to the antigen/MEC binding heterodimeric protein product of a vertebrate, e.g. mammalian, TCR gene complex, including the human TCR alpha, beta, gamma and delta chains. For example, the complete sequence of the human TCR beta locus has been sequenced, see, for example, Rowen et al. (1996) Science 272:1755-1762; the human TCR alpha locus has been sequenced and resequenced, see, for example, Mackelprang et al. (2006) Hum Genet. 119:255-266; and see, for example, Arden (1995) Immunogenetics 42:455-500 for a general analysis of the T-cell receptor V gene segment families; each of which is herein specifically incorporated by reference for the sequence information provided and referenced in the publication.

The term “antibody” or immunoglobulin” or “B cell receptor” or “BCR,” as used herein, is intended to refer to immunoglobulin molecules comprised of four polypeptide chains, two heavy (H) chains and two light (L) chains (lambda or kappa) inter-connected by disulfide bonds. An antibody has a known specific antigen with which it binds. Each heavy chain of an antibody is comprised of a heavy chain variable region (abbreviated herein as HCVR, HV or VH) and a heavy chain constant region. The heavy chain constant region is comprised of three domains, CH1, CH2 and CH3. Each light chain is comprised of a light chain variable region (abbreviated herein as LCVR or VL or KV or LV to designate kappa or lambda light chains) and a light chain constant region. The light chain constant region is comprised of one domain, CL. The heavy chain determines the class or isotype to which the immunoglobulin belongs. In mammals, for example, the five main immunoglobulin isotypes are IgA, IgD, IgG, IgE and IgM and they are classed according to the alpha, delta, epsilon, gamma or mu heavy chain they contain, respectively.

As noted, the diversity of the TCR and BCR chain CDRs is created by recombination of germline variable (V), diversity (D), and joining (J) gene segments, as well as by independent addition and deletion of nucleotides at each of the gene segment junctions during the process of TCR and BCR gene rearrangement. In the rearranged nucleic acid encoding a BCR heavy chain, CDR1 and CDR2 are found in the V gene segment and CDR3 includes some of the V gene segment and the D and J gene segments. In the rearranged nucleic acid encoding a BCR light chain, CDR1 and CDR2 are found in the V gene segment and CDR3 includes some of the V gene segment and the J gene segment. In the rearranged nucleic acid encoding a TCR beta and a TCR delta, for example, CDR1 and CDR2 are found in the V gene segments and CDR3 includes some of the V gene segment, and the D and J gene segments. In the rearranged nucleic acid encoding a TCR alpha and a TCR gamma, CDR1 and CDR2 are found in the V gene segments and CDR3 includes some of the V gene segment and the J gene segment.

In some embodiments, a multiplex amplification reaction is used to amplify cDNA derived from mRNA expressed from rearranged BCR and/or TCR genomic DNA. In some embodiments, a multiplex amplification reaction is used to amplify at least a portion of a BCR and/or TCR CDR from cDNA derived from a biological sample. In some embodiments, a multiplex amplification reaction is used to amplify at least two CDRs of a BCR and/or TCR from cDNA derived from a biological sample. In some embodiments, a multiplex amplification reaction is used to amplify at least three CDRs of a BCR and/or TCR from cDNA derived from a biological sample. In some embodiments, the resulting amplicons are used to determine the nucleotide sequences of the BCR and/or TCR CDRs expressed in the sample. In some embodiments, determining the nucleotide sequences of such amplicons comprising at least 3 CDRs is used to identify and characterize novel BCR and/or TCR alleles.

In some embodiments, a multiplex amplification reaction is used to amplify BCR and/or TCR genomic DNA having undergone V(D)J rearrangement. In some embodiments, a multiplex amplification reaction is used to amplify nucleic acid molecule(s) comprising at least a portion of a BCR and/or TCR CDR from gDNA derived from a biological sample. In some embodiments, a multiplex amplification reaction is used to amplify nucleic acid molecule(s) comprising at least two CDRs of a BCR and/or TCR from gDNA derived from a biological sample. In some embodiments, a multiplex amplification reaction is used to amplify nucleic acid molecules comprising at least three CDRs of a BCR and/or TCR from gDNA derived from a biological sample. In some embodiments, the resulting amplicons are used to determine the nucleotide sequences of the rearranged BCR and/or TCR CDRs in the sample. In some embodiments, determining the nucleotide sequences of such amplicons comprising at least CDR3 is used to identify and characterize novel BCR and/or TCR alleles

In some embodiments of the multiplex amplification reactions, each primer set used target a same BCR or TCR region however the different primers in the set permit targeting the gene's different V(D)J gene rearrangements. For example, the primer set for amplification of the expressed IgH or the rearranged IgH gDNA are all designed to target the same region(s) from IgH mRNA or IgH gDNA, respectively, but the individual primers in the set lead to amplification of the various IgH VDJ gene combinations. In some embodiments, at least one primer or primer set is directed to a relatively conserved region (e.g., a portion of the C gene) of an immune receptor gene and the other primer set includes a variety of primers directed to a more variable region of the same gene (e.g., a portion of the V gene). In other embodiments, at least one primer set includes a variety of primers directed to at least a portion of J gene segments of an immune receptor gene and the other primer set includes a variety of primers directed to at least a portion of V gene segments of the same gene.

In some embodiments, a multiplex amplification reaction is used to amplify cDNA derived from mRNA expressed from rearranged BCR genomic DNA, including rearranged IgH, IgK, and IgL genomic DNA. In some embodiments, at least a portion of a BCR CDR, for example CDR3, is amplified from cDNA in a multiplex amplification reaction. In some embodiments, at least two CDR portions of BCR are amplified from cDNA in a multiplex amplification reaction. In certain embodiments, a multiplex amplification reaction is used to amplify at least the CDR1, CDR2, and CDR3 regions of a BCR cDNA. In some embodiments, the resulting amplicons are used to determine the expressed BCR CDR nucleotide sequence. In some embodiments, the resulting amplicons are used to determine the expressed BCR CDR nucleotide sequence and Ig isotype of the sequence. In some embodiments, the resulting amplicons are used to determine the expressed IgH CDR nucleotide sequence and the Ig isotype and Ig sub-isotype.

In some embodiments, a multiplex amplification reaction is used to amplify rearranged BCR genomic DNA, including rearranged IgH, IgK, and IgL genomic DNA. In some embodiments, at least a portion of a BCR CDR, for example CDR3, is amplified from gDNA in a multiplex amplification reaction. In some embodiments, at least two CDR portions of BCR are amplified from gDNA in a multiplex amplification reaction. In certain embodiments, a multiplex amplification reaction is used to amplify at least the CDR1, CDR2, and CDR3 regions of a rearranged BCR gDNA. In some embodiments, the resulting amplicons are used to determine the rearranged BCR CDR nucleotide sequence. In some embodiments, the resulting amplicons are used to determine the rearranged BCR CDR nucleotide sequence and Ig isotype of the sequence.

In some embodiments, multiplex amplification reactions are performed with primer sets designed to generate amplicons which include the expressed CDR1, CDR2, and/or CDR3 regions of the target immune receptor mRNA. In some embodiments, multiplex amplification reactions are performed using (i) one set of primers in which each primer is directed to at least a portion of the framework region FR1 of a V gene and (ii) at least one primer directed to a portion of at least one C gene of the target immune receptor. In other embodiments, multiplex amplification reactions are performed using (i) one set of primers in which each primer is directed to at least a portion of the framework region FR2 of a V gene and (ii) at least one primer directed to a portion of at least one C gene of the target immune receptor. In other embodiments, multiplex amplification reactions are performed using (i) one set of primers in which each primer is directed to at least a portion of the framework region FR3 of a V gene and (ii) at least one primer directed to a portion of at least one C gene of the target immune receptor. In some embodiments, multiplex amplification reactions are performed with primer sets designed to generate amplicons which include one or more expressed IgH isotypes of the target mRNA and such reactions are performed using (i) one of the FR1, FR2, or FR3 primer sets noted above and (ii) a set of primers in which each primer is directed to a portion of at least one C gene of IgA, IgD, IgE, IgG, and/or IgM. In some embodiments, the C gene-directed primer(s) is directed C gene coding sequences within about 200 nucleotides of the 5′ end of the C gene(s). In some embodiments, the C gene-directed primer(s) is directed C gene coding sequences within about 150 nucleotides of the 5′ end of the C gene(s). In some embodiments, the C gene-directed primer(s) is directed C gene coding sequences within about 100 nucleotides of the 5′ end of the C gene(s). In some embodiments, the C gene-directed primer(s) is directed C gene coding sequences within about 50 nucleotides, within about 50 to about 150, within about 75 to about 175, or within about 100 to about 200 nucleotides of the 5′ end of the C gene(s). In some embodiments, the C gene-directed primer(s) is directed to C gene coding sequencing which not only distinguishes the isotype but also permits determination of a sub-isotype. For example, in some embodiments, the C gene-directed primer(s) generates sufficient portions of the constant region in the amplicon so that a sub-isotype can be determined based on the determined sequence data. In some embodiments, the C gene-directed primer(s) include primers which are directed to IgG and/or IgA C gene coding sequences which permit identification of IgG1, IgG2, IgG3, IgG4, IgA1, and IgA2 sub-isotypes.

In some embodiments, target-specific primers (e.g., the V gene FR1-, FR2- and FR3-directed primers, the J gene directed primers, and the C gene directed primers) used in the methods are selected or designed to satisfy any one or more of the following criteria: (1) includes two or more modified nucleotides within the primer sequence, at least one of which is included near or at the termini of the primer and at least one of which is included at, or about the center nucleotide position of the primer sequence; (2) length of about 15 to about 40 bases in length; (3) Tm of from above 60° C. to about 70° C.; (4) has low cross-reactivity with non-target sequences present in the sample of interest; (5) at least the first four nucleotides (going from 3′ to 5′ direction) are non-complementary to any sequence within any other primer present in the same reaction; and (6) non-complementarity to any consecutive stretch of at least 5 nucleotides within any other produced target amplicon. In some embodiments, the target-specific primers used in the methods provided are selected or designed to satisfy any 2, 3, 4, 5, or 6 of the above criteria.

In some embodiments, target amplicons using the amplification methods (and associated compositions, systems, and kits) disclosed herein, are used in the preparation of an immune receptor repertoire library. In some embodiments, the immune receptor repertoire library includes introducing adapter sequences to the termini of the target amplicon sequences. In certain embodiments, a method for preparing an immune receptor repertoire library includes generating target immune receptor amplicon molecules according to any of the multiplex amplification methods described herein, treating the amplicon molecule by digesting a modified nucleotide within the amplicon molecules' primer sequences, and ligating at least one adapter to at least one of the treated amplicon molecules, thereby producing a library of adapter-ligated target immune receptor amplicon molecules comprising the target immune receptor repertoire. In some embodiments, the steps of preparing the library are carried out in a single reaction vessel involving only addition steps. In certain embodiments, the method further includes clonally amplifying a portion of the at least one adapter-ligated target amplicon molecule.

In some embodiments, target amplicons using the methods (and associated compositions, systems, and kits) disclosed herein, are coupled to a downstream process, such as but not limited to, library preparation and nucleic acid sequencing. For example, target amplicons can be amplified using bridge amplification, emulsion PCR or isothermal amplification to generate a plurality of clonal templates suitable for nucleic acid sequencing. In some embodiments, the amplicon library is sequenced using any suitable DNA sequencing platform such as any next generation sequencing (NGS) platform, including semi-conductor sequencing technology.

In some embodiments, sequencing of immune receptor amplicons generated using the methods (and associated compositions and kits) disclosed herein, produces contiguous sequence reads from about 200 to about 600 nucleotides in length. In some embodiments, contiguous read lengths are from about 300 to about 400 nucleotides. In some embodiments, contiguous read lengths are from about 350 to about 450 nucleotides. In some embodiments, read lengths average about 300 nucleotides, about 350 nucleotides, or about 400 nucleotides. In some embodiments, contiguous read lengths are from about 250 to about 350 nucleotides, about 275 to about 340, or about 295 to about 325 nucleotides in length. In some embodiments, read lengths average about 270, about 280, about 290, about 300, or about 325 nucleotides in length. In other embodiments, contiguous read lengths are from about 180 to about 300 nucleotides, about 200 to about 290 nucleotides, about 225 to about 280 nucleotides, or about 230 to about 250 nucleotides in length. In some embodiments, read lengths average about 200, about 220, about 230, about 240, or about 250 nucleotides in length. In other embodiments, contiguous read lengths are from about 70 to about 200 nucleotides, about 80 to about 150 nucleotides, about 90 to about 140 nucleotides, or about 100 to about 120 nucleotides in length. In some embodiments, contiguous read lengths are from about 50 to about 170 nucleotides, about 60 to about 160 nucleotides, about 60 to about 120 nucleotides, about 70 to about 100 nucleotides, about 70 to about 90 nucleotides, or about 80 nucleotides in length. In some embodiments, read lengths average about 70, about 80, about 90, about 100, about 110, or about 120 nucleotides. In some embodiments, the sequence read length include the amplicon sequence and a barcode sequence. In some embodiments, the sequence read length does not include a barcode sequence.

In some embodiments, the amplification primers and primer pairs are target-specific sequences that can amplify specific regions of a nucleic acid molecule. In some embodiments, the target-specific primers can amplify expressed RNA or cDNA. In some embodiments, the target-specific primers can amplify mammalian RNA, such as human RNA or cDNA prepared therefrom, or murine RNA or cDNA prepared therefrom. In some embodiments, the target-specific primers can amplify DNA, such as gDNA. In some embodiments, the target-specific primers can amplify mammalian DNA, such as human DNA or murine DNA.

As described herein, RNA from a biological sample is converted to cDNA, typically using reverse transcriptase in a reverse transcription reaction, prior to the multiplex amplification. In some embodiments, a reverse transcription reaction is performed with the input RNA and a portion of the cDNA from the reverse transcription reaction is used in the multiplex amplification reaction. In some embodiments, substantially all of the cDNA prepared from the input RNA is added to the multiplex amplification reaction. In other embodiments, a portion, such as about 80%, about 75%, about 66%, about 50%, about 33%, or about 25% of the cDNA prepared from the input RNA is added to the multiplex amplification reaction. In other embodiments, about 15%, about 10%, about 8%, about 6%, or about 5% of the cDNA prepared from the input RNA is added to the multiplex amplification reaction.

In some embodiments, the amount of cDNA from a sample added to the multiplex amplification reaction can be about 0.001 ng to about 5 micrograms. In some embodiments, the amount of cDNA used for multiplex amplification of one or more immune repertoire target sequences can be from about 0.01 ng to about 2 micrograms. In some embodiments, the amount of cDNA used for multiplex amplification of one or more target sequences can be from about 0.1 ng to about 1 microgram or about 1 ng to about 0.5 microgram. In some embodiments, the amount of cDNA used for multiplex amplification of one or more immune repertoire target sequences is about 0.5 ng, about 1 ng, about 5 ng, about 10 ng, about 25 ng, about 50 ng, about 100 ng, about 200 ng, about 250 ng, about 500 ng, about 750 ng, or about 1000 ng. In some embodiments, the amount of cDNA used for multiplex amplification of one or more immune repertoire target sequences is from about 0.01 ng to about 10 ng cDNA, from about 0.05 ng to about 5 ng cDNA, from about 0.1 ng to about 2 ng cDNA, or from about 0.01 ng to about 1 ng cDNA. In some embodiments, the amount of cDNA used for multiplex amplification of one or more immune repertoire target sequences is about 0.005 ng, about 0.01 ng, about 0.05 ng, about 0.1 ng, about 0.2 ng, about 0.5 ng, about 1.0 ng, about 2.0 ng, or about 5.0 ng.

In some embodiments, mRNA is obtained from a biological sample and converted to cDNA for amplification purposes using conventional methods. Methods and reagents for extracting or isolating nucleic acid from biological samples are well known and commercially available. In some embodiments, RNA extraction from biological samples is performed by any method described herein or otherwise known to those of skill in the art, e.g., methods involving proteinase K tissue digestion and alcohol-based nucleic acid precipitation, treatment with DNAse to digest contaminating DNA, and RNA purification using silica-gel-membrane technology, or any combination thereof. Exemplary methods for RNA extraction from biological samples using commercially available kits including RECOVERALL™ Multi-Sample RNA/DNA Workflow (Invitrogen), RECOVERALL™ Total Nucleic Acid Isolation Kit (Invitrogen), NUCLEOSPIN® RNA blood (Macherey-Nagel), PAXGENE® Blood RNA system, TRI REAGENT™ (Invitrogen), PURELINK™ RNA Micro Scale kit (Invitrogen), MAGMAX™ FFPE DNA/RNA Ultra Kit (Applied Biosystems) ZR RNA MICROPREP™ kit (Zymo Research), RNeasy Micro kit (Qiagen), and RELIAPREP™ RNA Tissue miniPrep system (Promega).

A sample or biological sample, as used herein, refers to a composition from an individual that contains or may contain cells related to the immune system. Exemplary biological samples, include without limitation, tissue (for example, lymph node, organ tissue, bone marrow), whole blood, synovial fluid, cerebral spinal fluid, tumor biopsy, and other clinical specimens containing cells. The sample may include normal and/or diseased cells and be a fine needle aspirate, fine needle biopsy, core sample, or other sample. In some embodiments, the biological sample may comprise hematopoietic cells, peripheral blood mononuclear cells (PBMCs), T cells, B cells, tumor infiltrating lymphocytes (“TILs”) or other lymphocytes. In some embodiments, the sample may be fresh (e.g., not preserved), frozen, or formalin-fixed paraffin-embedded tissue (FFPE). Some samples comprise cancer cells, such as carcinomas, melanomas, sarcomas, lymphomas, myelomas, leukemias, and the like, and the cancer cells may be circulating tumor cells. In some embodiments, the biological sample comprises cfDNA, such as found, for example, in blood or plasma.

The biological sample can be a mix of tissue or cell types, a preparation of cells enriched for at least one particular category or type of cell, or an isolated population of cells of a particular type or phenotype. Samples can be separated by centrifugation, elutriation, density gradient separation, apheresis, affinity selection, panning, FACS, centrifugation with Hypaque, etc. prior to analysis. Methods for sorting, enriching for, and isolating particular cell types are well-known and can be readily carried out by one of ordinary skill. In some embodiments, the sample may a preparation enriched for B cells.

In some embodiments, the provided methods and systems include processes for analysis of immune repertoire receptor cDNA or gDNA sequence data and for identification and/or removing PCR or sequencing-derived error(s) from the determined immune receptor sequence.

In some embodiments, the error correction strategy includes the following steps:
1) Align the sequenced rearrangement to a reference database of variable, diversity and joining/constant genes to produce a query sequence/reference sequence pair. Many alignment procedures may be used for this purpose including, for example, IgBLAST, a freely-available tool from the NCBI, and custom computer scripts.
2) Realign the reference and query sequences to each other, taking into account the flow order used for sequencing. The flow order provides information that allows one to identify and correct some types of erroneous alignments.
3) Identify the borders of the CDR3 region by their characteristic sequence motifs.
4) Over the aligned portion of the rearrangement corresponding to the variable gene and joining/constant genes, excluding the CDR3 region, identify indels in the query with respect to the reference and alter the mismatching query base position so that it is consistent with the reference.
5) For the CDR3 region, if the CDR3 length is not a multiple of three (indicative of an indel error):
   (a) Search the CDR3 for the homopolymer stretch having the highest probability of containing a sequence error, based on PHRED score (denoted e).
   (b) Obtain the probability of error over the entire CDR3 region based on PHRED score (denoted t)
   (c) If e/t is greater than a defined threshold, edit the homopolymer by either increasing or decreasing the length of the homopolymer by one base such that the CDR3 nucleotide length is a multiple of three.
   (d) As an alternative to steps a-c, search the CDR3 for the longest homopolymer, and if the length of the homopolymer is above a defined threshold, edit the homopolymer by either increasing or decreasing the length of the homopolymer by one base such that the CDR3 nucleotide length is a multiple of three.

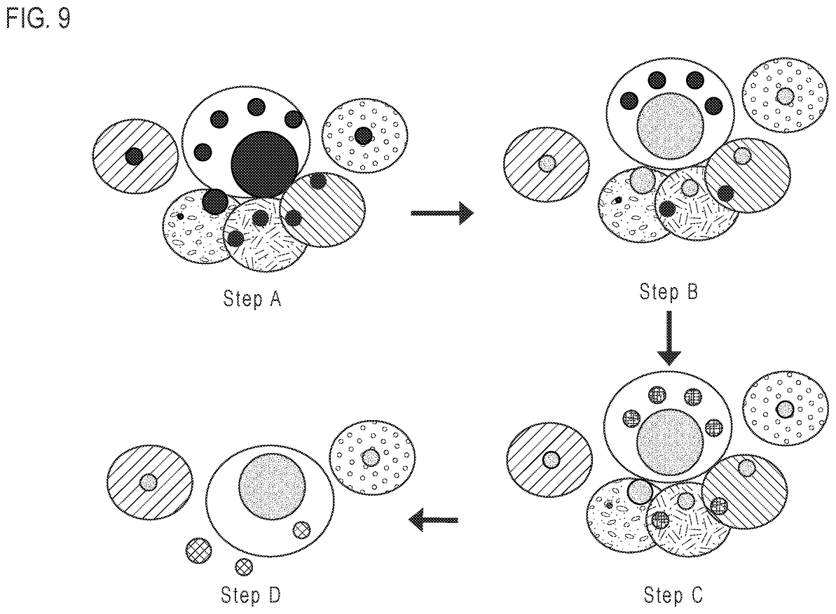

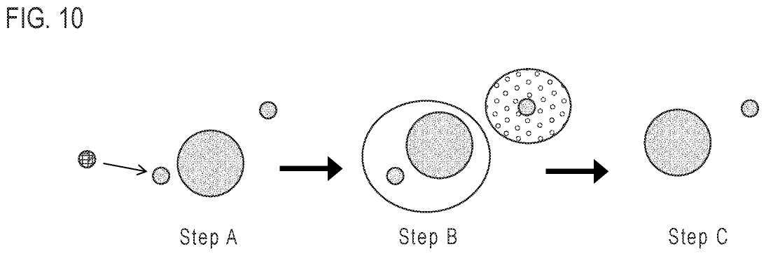

In some embodiments, methods are provided to identify B cell and/or T cell clones in repertoire data that are robust to PCR and sequencing error. Accordingly, the following describes steps that may be employed in such methods to identify B cell and/or T cell clones in a manner that is robust to PCR and sequencing error. Table 1 is a diagram of an exemplary workflow for use in identifying and removing PCR or sequencing-derived errors from immune receptor sequencing data. Exemplary portions and embodiments of this workflow are also represented in FIGS. 9-10.

TABLE 1
SEQUENCE CORRECTION WORKFLOW

| A. | Raw bam file |
|---|---|
| B. C. | 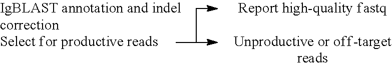 |
| D. E. F. | 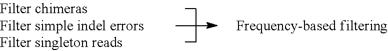 |
| G. | Filter truncated reads |
| H. | Filter for rearrangements |
| | with bidirectional support |

| | |
|---|---|
| I. | 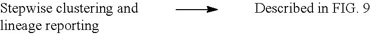 |

For a set of TCR or BCR sequences derived from mRNA or gDNA, where 1) each sequence has been annotated as a productive rearrangement, either natively or after error correction, such as previously described, and 2) each sequence has an identified V gene and CDR3 nucleotide region, in some embodiments, methods include the following:
1) Identify and exclude chimeric sequences. For each unique CDR3 nucleotide sequence present in the dataset, tally the number of reads having that CDR3 nucleotide sequence and any of the possible V genes. Any V gene-CDR3 combination making up less than 10% of total reads for that CDR3 nucleotide sequence is flagged as a chimera and eliminated from downstream analyses. As an example, for the sequences below having the same CDR3 nucleotide sequence, e.g., the sequences having TRBV3 and TRBV6 paired with CDR3nt sequence AATTGGT will be flagged as chimeric.

| V gene | CDR3nt | Read counts |
|---|---|---|
| TRBV2 | AATTGGT | 1000 |
| TRBV3 | AATTGGT | 10 |
| TRBV6 | AATTGGT | 3 |

2) Identify and exclude sequences containing simple indel errors. For each read in the dataset, obtain the homopolymer-collapsed representation of the CDR3 sequence of that read. For each set of reads having the same V gene and collapsed-CDR3 combination, tally the number of occurrences of each non-collapsed CDR3 nucleotide sequence. Any non-collapsed CDR3 sequence making up <10% of total reads for that read set is flagged as having a simple homopolymer error. As an example, three different V gene-CDR3 nucleotide sequences are presented that are identical after homopolymer collapsing of the CDR3 nucleotide sequence. The two less frequent V gene-CDR3 combinations make up <10% of total reads for the read set and will be flagged as containing a simple indel error. For example:

| | | Homopolymer | |
|---|---|---|---|
| | | collapsed | Read |
| V gene | CDR3nt | CDR3 nt | counts |
| TRBV2 | AATTGGT | ATGT | 1000 |
| TRBV2 | AAATGGT | ATGT | 10 |
| TRBV2 | AAAATTT | ATGT | 3 |
| | GGT | | |
| | (SEQ ID | | |
| | NO: 1) | | |

3) Identify and exclude singleton reads. For each read in the dataset, tally the number of times that the exact read sequence is found in the dataset. Reads that appear only once in the dataset will be flagged as singleton reads.
4) Identify and exclude truncated reads. For each read in the dataset, determine whether the read possesses an annotated V gene FR1, CDR1, FR2, CDR2, and FR3 region, as indicated by the IgBLAST alignment of the read to the IgBLAST reference V gene set. Reads that do not possess the above regions are flagged as truncated if the region(s) is expected based on the particular V gene primer used for amplification.
5) Identify and exclude rearrangements lacking bidirectional support. For each read in the dataset, obtain the V gene and CDR3 sequence of the read as well as the strand orientation of the read (plus or minus strand). For each V gene-CDR3 combination in the dataset, tally the number of plus and minus strand reads having that V gene-CDR3nt combination. V gene-CDR3nt combinations that are only present in reads of one orientation will be deemed to be a spurious. All reads having a spurious V gene-CDR3nt combination will be flagged as lacking bidirectional support.
6) For genes that have not been flagged, perform stepwise clustering based on CDR3 nucleotide similarity. Separate the sequences into groups based on the V gene identity of the read, excluding allele information (v-gene groups). For each group:
   a. Arrange reads in each group into clusters using cd-hit-est and the following parameters:
      cd-hit-est vgene_groups.fa-o clustered_vgene_groups.cdhit-T24-d 0-M 100000-B 0-r 0-g 1-S0-U 2-uL 0.05-n 10-l7. (The freely available software program cd-hit-est clusters a nucleotide dataset into clusters that meet a user-defined similarity threshold. (For code and instructions on cd-hit-est, see www.github.com/weizhongli/cdhit/wiki/3.-User %27s-Guide#CDHITEST).
      Where vgene_groups.fa is a fasta format file of the CDR3 nucleotide regions of sequences having the same V gene and clustered_vgene_groups.cdhit is the output, containing the subdivided sequences.
   b. Assign each sequence in a cluster the same clone ID, used to denote that members of the subgroup are believed to represent the same T cell clone or B cell clone.
   c. Chose a representative sequence for each cluster, such that the representative sequence is the sequence that appears the greatest number of times, or, in cases of a tie, is randomly chosen.
   d. Merge all other reads in the cluster into the representative sequence such that the number of reads for the representative sequence is increased according to the number of reads for the merged sequences.
   e. Compare the representative sequences within a v-gene group to each other on the basis of hamming distance. If a representative sequence is within a hamming distance of 1 to a representative sequence that is >50 times more abundant, merge that sequence into the more common representative sequence. If a representative sequence is within a hamming distance of 2 to a representative sequence that is >10000 times more abundant, merge that sequence into the more common representative sequence.
   f. Identify complex sequence errors. Homopolymer-collapse the representative sequences within each V gene group, then compare to each other using Levenshtein distances. If a representative sequence is within a Levenshtein distance of 1 to a representative sequence that is >50 times more abundant, merge that sequence into the more common representative sequence.
   g. Identify CDR3 misannotation errors. Homopolymer-collapse the representative sequences within each V gene group, then perform a pairwise comparison of each homopolymer-collapsed sequence. For each pair of sequences, determine whether one sequence is a subset of the other sequence. If so, merge the less abundant sequence into the more abundant sequence if the more abundance sequence is >500 fold more abundant.
7) Report cluster representatives to user.

In some embodiments, step 6 of the above workflow separates the rearrangement sequences into groups based on the V-gene identity (excluding allele information), and the CDR3 nucleotide length. In other embodiments, the J-gene identity and/or isotype identity is also used as part of the grouping criteria. Accordingly, in some embodiments, step 6 of the above workflow includes the following steps:
a. Arrange reads in each group into clusters using cd-hit-est and the following parameters:
   cd-hit-est-i vgene_groups.fa-o clustered_vgene_groups.cdhit-T24-l9-d 0-M 100000-B 0-r 0-g 1-S15-U 2-uL 0.05-n9.
   Where vgene_groups.fa is a fasta format file of the sequenced portion of the VDJ rearrangement.
   In some embodiments, the full sequence of the VDJ is considered for clustering as somatic hypermutation may occur throughout the VDJ region.
b. Assign each sequence in a cluster the same clone ID, used to denote that members of the subgroup are believed to represent the same T cell clone or B cell clone.
c. Chose a representative sequence for each cluster, such that the representative sequence is the sequence that appears the greatest number of times, or, in cases of a tie, is randomly chosen.
d. Merge all other reads in the cluster into the representative sequence such that the number of reads for the representative sequence is increased according to the number of reads for the merged sequences.
e. Compare the representative sequences within a v-gene group to each other on the basis of hamming distance. If a representative sequence is within a hamming distance of 1 to a representative sequence that is >50 times more abundant, merge that sequence into the more common representative sequence. If a representative sequence is within a hamming distance of 2 to a representative sequence that is >10000 times more abundant, merge that sequence into the more common representative sequence. In some embodiments, fold thresholds of >50/3 and >10000/3, among others are used to merge sequences of hamming distances 1 or 2, respectively. Reducing the fold thresholds can be useful when comparing sequences of the entire VDJ region rather than sequences of only the CDR3 region as the longer sequence has a greater chance of accumulating amplification and/or sequencing errors.
f Identify complex sequence errors. Homopolymer-collapse the representative sequences within each V gene group, then compare to each other using Levenshtein distances. If a representative sequence is within a Levenshtein distance of 1 to a representative sequence that is >50 times more abundant, merge that sequence into the more common representative sequence.
g. Identify CDR3 misannotation errors. Homopolymer-collapse the representative sequences within each V gene group, then perform a pairwise comparison of each homopolymer-collapsed sequence. For each pair of sequences, determine whether one sequence is a subset of the other sequence. If so, merge the less abundant sequence into the more abundant sequence if the more abundance sequence is >500 fold more abundant.

In some embodiments, the provided workflows are not limited to the frequency ratio thresholds listed in the various steps, and other frequency ratio thresholds may be substituted for the representative frequency ratio thresholds included above. The frequency ratio refers to a ratio of the abundance value of the more common representative sequence to the abundance value of the less common representative sequence. The frequency ratio threshold gives the threshold at which the less common representative sequence is merged into the more common representative sequence. For example, in some embodiments, comparing the representative sequences within a v-gene group to each other on the basis of hamming distance may use a frequency ratio threshold other than those listed in step (e) above. For example and without limitation, frequency ratio thresholds of 1000, 5000, 20,000, etc. may be used if a representative sequence is within a hamming distance of 2 to a representative sequence. For example and without limitation, frequency ratio thresholds of 20, 100, 200, etc. may be used if a representative sequence is within a hamming distance of 1 to a representative sequence. The frequency ratio thresholds provided are representative of the general process of labeling the more abundant sequence of a similar pair as a correct sequence.

Similarly, when comparing the frequencies of two sequences at other steps in the workflows, e.g., step (1), step (2), step (6f) and step (6g), frequency ratio thresholds other than those listed in the step above may be used.

As used herein, the term “homopolymer-collapsed sequence” is intended to represent a sequence where repeated bases are collapsed to a single base representative.

As used herein, the terms “clone,” “clonotype,” “lineage,” or “rearrangement” are intended to describe a unique V gene nucleotide combination for an immune receptor, such as a TCR or BCR. For example, a unique V gene-CDR3 nucleotide combination.

As used herein, the term “productive reads” refers to a TCR or BCR sequence reads that have no stop codon and have in-frame variable gene and joining gene segments. Productive reads are biologically plausible in coding for a polypeptide.

As used herein, “chimeras” or chimeric sequences” refer to artefactual sequences that arise from template switching during target amplification, such as PCR. Chimeras typically present as a CDR3 sequence grafted onto an unrelated V gene, resulting in a CDR3 sequence that is associated with multiple V genes within a dataset. The chimeric sequence is usually far less abundant than the true sequence in the dataset.

As used herein, the term “indel” refers to an insertion and/or deletion of one or more nucleotide bases in a nucleic acid sequence. In coding regions of a nucleic acid sequence, unless the length of an indel is a multiple of 3, it will produce a frameshift when the sequence is translated. As used herein, “simple indel errors” are errors that do not alter the homopolymer-collapsed representation of the sequence. As used herein, “complex indel errors” are indel sequencing errors that alter the homopolymer-collapsed representation of the sequence and include, without limitation, errors that eliminate a homopolymer, insert a homopolymer into the sequence, or create a dyslexic-type error.

As used herein, “singleton reads” refer to sequence reads whose indel-corrected sequence appears only once in a dataset. Typically, singleton reads are enriched for reads containing a PCR or sequencing error.

As used herein, “truncated reads” refer to immune receptor sequence reads that are missing annotated V gene regions. For example, truncated reads include, without limitation, sequence reads that are missing annotated TCR or BCR V gene FR1, CDR1, FR2, CDR2, or FR3 regions. Such reads typically are missing a portion of the V gene sequence due to quality trimming. Truncated reads can give rise to artifacts if the truncation leads one to misidentify the V gene.

In the context of identified V gene-CDR3 sequences (clonotypes), “bidirectional support” indicates that a particular V gene-CDR3 sequence is found in at least one read that maps to the plus strand (proceeding from the V gene to constant gene) and at least one reads that maps to the minus strand (proceeding form the constant gene to the V gene). Systematic sequencing errors often lead to identification of V gene-CDR3 sequences having unidirectional support.

For a set of sequences that have been grouped according to a predetermined sequence similarity threshold to account for variation due to PCR or sequencing error, the “cluster representative” is the sequence that is chosen as most likely to be error free. This is typically the most abundant sequence.

As used herein, “IgBLAST annotation error” refers to rare events where the border of the CDR3 is identified to be in an incorrect adjacent position. These events typically add three bases to the 5′ or 3′ end of a CDR3 nucleotide sequence.

For two sequences of equal length, the “Hamming distance” is the number of positions at which the corresponding bases or amino acids are different. For any two sequences, the “Levenshtein distance” or the “edit distance” is the number of single base or amino acid edits required to make one nucleotide or amino acid sequence into another nucleotide or amino acid sequence.

In certain embodiments, methods comprise the use of target immune receptor primer sets wherein the primers are directed to sequences of the same target immune receptor gene, e.g., BCR (immunoglobulin) and TCR genes. In some embodiments the immune receptor is an antibody receptor selected from the group consisting of heavy chain alpha, heavy chain delta, heavy chain epsilon, heavy chain gamma, heavy chain mu, light chain kappa, and light chain lambda. In some embodiments a T cell receptor is a T cell receptor selected from the group consisting of TCR alpha, TCR beta, TCR gamma, and TCR delta. In some embodiments, methods comprise the use of target immune receptor primer sets wherein at least one of the primer sets is directed to sequences of a BCR and another primer set is directed to sequences of a TCR, and both the BCR and TCR target nucleic acids from a sample are amplified in a single multiplex amplification reaction.

In certain embodiments, provided is a method for amplification of expression nucleic acid sequences of a BCR repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of a framework region within the V gene, and ii) one or more C gene primers directed to at least a portion of the respective target constant gene of the BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region.

In certain embodiments, provided is a method for amplification of expression nucleic acid sequences of an immune receptor repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of framework region 1 (FR1) within the V gene, and ii) one or more C gene primers directed to at least a portion of the respective target C gene of the BCR coding sequence, wherein each set of i) and ii) primers directed to the same target immune receptor sequences is selected from the group consisting of IgH, IgL, and IgK and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the framework region 1 of the template molecules. In certain embodiments the one or more C gene primers of ii) comprises at least two primers that anneal to at least a portion of a C gene portion of the template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules. In particular embodiments at least one set of the generated amplicons includes complementarity determining regions CDR1, CDR2, and CDR3 of a BCR expression sequence. In some embodiments the amplicons are about 300 to about 600 nucleotides in length or at least about 350 to about 500 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of the respective target C gene(s) of the BCR coding sequence, wherein each set of i) and ii) primers directed to the same target immune receptor sequences is selected from the group consisting of IgH, an IgL, and an IgK, thereby generating BCR amplicon molecules. Sequencing of resulting BCR amplicon molecules is then performed and the sequences of the BCR amplicon molecules determined thereby provides sequence of the BCR repertoire in the sample. In particular embodiments, determining the sequence of the BCR amplicon molecules includes obtaining initial sequence reads, aligning the initial sequence read to a reference sequence and identifying a productive reads, correcting one or more indel errors to generate rescued productive sequence reads; and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 300 and 600 nucleotides, or is between 350 and 550 nucleotides, or is between 330 and 425 nucleotides, or is about 350 to about 425 nucleotides, depending in part on inclusion of any barcode sequence in the read length. In certain embodiments at least one set of the sequenced amplicons includes complementarity determining regions CDR1, CDR2, and CDR3 of a BCR expression sequence.

In some embodiments, methods provided utilize target BCR primer sets comprising V gene primers wherein the one or more of a plurality of V gene primers are directed to sequences over an FR1 region about 70 nucleotides in length. In other particular embodiments the one or more of a plurality of V gene primers are directed to sequences over an FR1 region about 50 nucleotides in length. In certain embodiments a target BCR primer set comprises V gene primers comprising about 18 to about 45 different FR1-directed primers. In some embodiments a target BCR primer set comprises V gene primers comprising about 22 to about 35 different FR1-directed primers. In some embodiments a target BCR primer set comprises V gene primers comprising about 25 to about 35 different FR1-directed primers. In certain embodiments a target BCR primer set comprises V gene primers comprising about 40 to about 65 different FR1-directed primers. In some embodiments a target BCR primer set comprises V gene primers comprising about 48 to about 60 different FR1-directed primers. In some embodiments the target BCR primer set comprises one or more C gene primers. In particular embodiments a target immune receptor primer set comprises at least 5 to about 15 C gene primers wherein each is directed to at least a portion of the same 50 nucleotide region within each of the target C genes. In particular embodiments a target immune receptor primer set comprises at least 2 to about 8 C gene primers wherein each is directed to at least a portion of the same 50 nucleotide region within each of the target C genes. In some embodiments a target BCR primer set comprises two or more C gene primers directed to different Ig isotype molecules, e.g., IgA, IgD, IgG, IgM and IgE. In some embodiments a target BCR primer set comprises at least five C gene primers each primer directed to a C gene of a different Ig isotype molecule.

In certain embodiments, provided is a method for amplification of expression nucleic acid sequences of a BCR repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of framework region 3 (FR3) within the V gene, and ii) one or more C gene primers directed to at least a portion of the respective target C gene of the BCR coding sequence, wherein each set of i) and ii) primers directed to the same target immune receptor sequences is selected from the group consisting of IgH, an IgL, and an IgK, and wherein performing amplification using each set results in amplicons representing the entire repertoire of the respective immune receptor in the sample; thereby generating immune receptor amplicons comprising the BCR repertoire. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In more particular embodiments the one or more plurality of V gene primers of i) are directed to sequences over about a 40 to about a 60 nucleotide portion of the framework region. In some embodiments the one or more plurality of V gene primers of i) anneal to at least a portion of the framework 3 region of the template molecules. In certain embodiments the one or more C gene primers of ii) comprises at least two primers that anneal to at least a portion of the C gene of the BCR template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules. In particular embodiments at least one set of the generated amplicons includes complementarity determining region CDR3 of a BCR expression sequence. In some embodiments the amplicons are about 80 to about 200 nucleotides in length, about 80 to about 140 nucleotides in length, about 90 to about 130 nucleotides in length or at least about 100 to about 120 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of framework region 3 (FR3) within the V gene, and ii) one or more C gene primers directed to at least a portion of the respective target C gene(s) of the BCR coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the BCR amplicon molecules determined thereby provides sequence of the BCR in the sample. In particular embodiments, determining the sequence of the BCR amplicon molecules includes obtaining initial sequence reads, aligning the initial sequence read to a reference sequence and identifying a productive reads, correcting one or more indel errors to generate rescued productive sequence reads; and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified BCR repertoire are compared to a contemporaneous or current version of the IMGT database and the sequence of at least one allelic variant absent from that IMGT database is identified. In some embodiments the average sequence read length is between 80 and 185 nucleotides, is between 115 and 200 nucleotides, is between 90 and 130 nucleotides, or is between about 100 and about 120 nucleotides, depending in part on inclusion of any barcode sequence in the read length. In certain embodiments at least one set of the sequenced amplicons includes complementarity determining region CDR3 of a BCR expression sequence.

In certain embodiments, provided is a method for amplification of expression nucleic acid sequences of a BCR repertoire in a sample, comprising performing a multiplex amplification reaction to amplify BCR nucleic acid template molecules having a constant portion and a V gene portion using at least one set of: i) a plurality of V gene primers directed to a majority of different V genes of at least one BCR coding sequence comprising at least a portion of framework region 2 (FR2) within the V gene, and ii) one or more C gene primers directed to at least a portion of the C gene of the respective BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating amplicons comprising the BCR repertoire. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the FR2 region of the BCR template molecules. In certain embodiments the one or more C gene primers of ii) comprises at least two primers that anneal to at least a portion of the constant portion C gene of the BCR template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules. In particular embodiments at least one set of the generated amplicons includes complementarity determining regions CDR2 and CDR3 of a BCR expression sequence. In some embodiments the amplicons are about 180 to about 375 nucleotides in length, about 200 to about 350 nucleotides, about 225 to about 325 nucleotides, or about 250 to about 300 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of FR2 within the V gene, and ii) one or more C gene primers directed to at least a portion of the respective target C gene of the BCR coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the BCR amplicon molecules determined thereby provides sequence of the BCR repertoire in the sample. In particular embodiments, determining the sequence of the BCR amplicon molecules includes obtaining initial sequence reads, aligning the initial sequence read to a reference sequence and identifying productive reads, correcting one or more indel errors to generate rescued productive sequence reads; and determining the sequences of the resulting BCR molecules. In particular embodiments the combination of productive reads and rescued productive reads is at least 40%, at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between about 200 and about 375 nucleotides, between about 250 and about 350 nucleotides, or between about 275 and about 350 nucleotides, depending in part on inclusion of any barcode sequence in the read length. In certain embodiments at least one set of the sequenced amplicons includes complementarity determining regions CDR2 and CDR3 of a BCR expression sequence.

In particular embodiments, methods provided utilize target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers comprising about 4 to about 20 different FR2-directed primers. In some embodiments a target BCR primer set comprises V gene primers comprising about 5 to about 15 different FR2-directed primers. In some embodiments a target BCR primer set comprises V gene primers comprising about 5, 6, 7, 8, 9, 10, 11, or 12 different FR2-directed primers. In some embodiments the target BCR primer set comprises one or more C gene primers. In particular embodiments a target immune receptor primer set comprises at least 5 to about 15 C gene primers wherein each is directed to at least a portion of the same 50 nucleotide region within each of the target C genes. In particular embodiments a target BCR primer set comprises at least 2 to about 8 C gene primers wherein each is directed to at least a portion of the same 50 nucleotide region within each of the target C genes. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of a BCR coding sequence comprising at least a portion of a framework region within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In particular embodiments the one or more plurality of J gene primers of ii) are directed to sequences over about a 50 nucleotide portion of the J gene. In more particular embodiments the one or more plurality of J gene primers of ii) are directed to sequences over about a 30 nucleotide portion of the J gene. In certain embodiments, the one or more plurality of J gene primers of ii) are directed to sequences completely within the J gene.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating BCR amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In more particular embodiments the one or more plurality of V gene primers of i) are directed to sequences over about a 40 to about a 60 nucleotide portion of the framework region. In some embodiments the one or more plurality of V gene primers of i) anneal to at least a portion of the framework 3 region of the template molecules. In certain embodiments the plurality of J gene primers of ii) comprises at least two primers that anneal to at least a portion of the J gene portion of the template molecules. In some embodiments the plurality of J gene primers of ii) comprises at least 2 to about 8 primers that anneal to at least a portion of the J gene portion of the template molecules. In some embodiments the plurality of J gene primers of ii) comprises about 4 primers that anneal to at least a portion of the J gene portion of the template molecules. In some embodiments the plurality of J gene primers of ii) comprises about 3 to about 6 primers that anneal to at least a portion of the J gene portion of the template molecules. In particular embodiments at least one set of the generated amplicons includes complementarity determining region CDR3 of a BCR expression sequence. In some embodiments the amplicons are about 60 to about 160 nucleotides in length, about 70 to about 100 nucleotides in length, about 100 to about 120 nucleotides in length, at least about 70 to about 90 nucleotides in length, about 80 to about 90 nucleotides in length, or about 80 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the immune receptor amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified BCR repertoire are compared to a contemporaneous or current version of the IMGT database and the sequence of at least one allelic variant absent from that IMGT database is identified. In some embodiments the sequence read lengths are about 60 to about 185 nucleotides, depending in part on inclusion of any barcode sequence in the read length. In some embodiments the average sequence read length is between 90 and 120 nucleotides, is between 70 and 90 nucleotides, or is between about 75 and about 85 nucleotides, or is about 80 nucleotides. In certain embodiments at least one set of the sequenced amplicons includes complementarity determining region CDR3 of a BCR expression sequence.

In particular embodiments, methods provided utilize target BCR primer sets comprising V gene primers wherein the one or more of a plurality of V gene primers are directed to sequences over an FR3 region about 50 nucleotides in length. In other embodiments the one or more of a plurality of V gene primers are directed to sequences over an FR3 region about 70 nucleotides in length. In other particular embodiments the one or more of a plurality of V gene primers are directed to sequences over an FR3 region about 40 to about 60 nucleotides in length. In certain embodiments a target BCR primer set comprises V gene primers comprising about 50 to about 85 different FR3-directed primers. In certain embodiments a target BCR primer set comprises V gene primers comprising about 55 to about 80 different FR3-directed primers. In some embodiments, a target immune receptor primer set comprises V gene primers comprising about 62 to about 75 different FR3-directed primers. In some embodiments, a target BCR primer set comprises V gene primers comprising about 65, 66, 67, 68, 69, or 70 different FR3-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of framework region 1 (FRI) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules. Sequencing of resulting immune receptor amplicon molecules is then performed and the sequences of the BCR amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 50%, at least 60% at least 70% or at least 75% of the sequencing reads for the immune receptors. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 200 and 350 nucleotides, between 225 and 325 nucleotides, between 250 and 300 nucleotides, between 270 and 300 nucleotides, or is between 295 and 325 nucleotides, depending in part on inclusion of any barcode sequence in the read length. In certain embodiments at least one set of the sequenced amplicons includes complementarity determining regions CDR1, CDR2, and CDR3 of a BCR expression sequence.

In particular embodiments, methods provided utilize target BCR primer sets comprising V gene primers wherein the one or more of a plurality of V gene primers are directed to sequences over an FR1 region about 70 nucleotides in length. In other certain embodiments the one or more of a plurality of V gene primers are directed to sequences over an FR1 region about 80 nucleotides in length. In other particular embodiments the one or more of a plurality of V gene primers are directed to sequences over an FR1 region about 50 nucleotides in length. In certain embodiments a target BCR primer set comprises V gene primers comprising about 18 to about 45 different FR1-directed primers. In some embodiments a target BCR primer set comprises V gene primers comprising about 22 to about 35 different FR1-directed primers. In some embodiments a target BCR primer set comprises V gene primers comprising about 25 to about 35 different FR1-directed primers. In certain embodiments a target BCR primer set comprises V gene primers comprising about 40 to about 65 different FR1-directed primers. In some embodiments a target BCR primer set comprises V gene primers comprising about 48 to about 60 different FR1-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of a BCR coding sequence comprising at least a portion of framework region 2 (FR2) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the FR2 region of the template molecules. In certain embodiments the plurality of J gene primers of ii) comprise at least ten primers that anneal to at least a portion of the J gene of the template molecules. In some embodiments the plurality of J gene primers of ii) comprises about 14 primers that anneal to at least a portion of the J gene portion of the template molecules. In some embodiments the plurality of J gene primers of ii) at least two primers that anneal to at least a portion of the J gene portion of the template molecules. In some embodiments the plurality of J gene primers of ii) comprises at least 2 to about 8 primers that anneal to at least a portion of the J gene portion of the template molecules. In some embodiments the plurality of J gene primers of ii) comprises about 4 primers that anneal to at least a portion of the J gene portion of the template molecules. In some embodiments the plurality of J gene primers of ii) comprises about 3 to about 6 primers that anneal to at least a portion of the J gene portion of the template molecules. In particular embodiments at least one set of the generated amplicons includes complementarity determining regions CDR2 and CDR3 of a BCR gene sequence. In some embodiments the amplicons are about 160 to about 270 nucleotides in length, about 180 to about 250 nucleotides, or about 195 to about 225 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of FR2 within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules. Sequencing of resulting immune receptor amplicon molecules is then performed and the sequences of the BCR amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 40%, at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 160 and 300 nucleotides, between 180 and 280 nucleotides, between 200 and 260 nucleotides, or between 225 and 270 nucleotides, depending in part on inclusion of any barcode sequence in the read length. In certain embodiments at least one set of the sequenced amplicons includes complementarity determining regions CDR2 and CDR3 of a BCR expression sequence.

In particular embodiments, methods provided utilize target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers comprising about 4 to about 20 different FR2-directed primers. In some embodiments a target BCR primer set comprises V gene primers comprising about 5 to about 15 different FR2-directed primers. In some embodiments a target BCR primer set comprises V gene primers comprising about 5, 6, 7, 8, 9, 10, 11, or 12 different FR2-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In some embodiments, methods, compositions, and systems are provided for determining the immune repertoire of a biological sample by assessing both expressed immune receptor RNA and rearranged immune receptor genomic DNA (gDNA) from a biological sample. In some embodiments, the sample RNA and gDNA may be assessed concurrently and following reverse transcription of the RNA to form cDNA, the cDNA and gDNA may be amplified in the same multiplex amplification reaction. In some embodiments, cDNA from the sample RNA and the sample gDNA may undergo multiplex amplification in separate reactions. In some embodiments, cDNA from the sample RNA and sample gDNA may undergo multiplex amplification with parallel primer pools. In some embodiments, the same BCR-directed primer pools are used to assess the BCR repertoire of gDNA and RNA from the sample. In some embodiments, different immune receptor-directed primer pools are used to assess the immune repertoire of gDNA and RNA from the sample. In some embodiments, multiplex amplification reactions are performed separately with cDNA from the sample RNA and with sample gDNA to amplify the same or different target immune receptor molecules from the sample and the resulting immune receptor amplicons are sequenced, thereby providing sequence of the expressed immune receptor RNA and rearranged immune receptor gDNA of a biological sample.

In some embodiments, different immune receptor-directed primer pools are used to assess the immune repertoire of gDNA and/or RNA from the sample. In some embodiments, multiplex amplification reactions are performed with a set of IgH primers provided herein and with a set of TCR beta-directed primers, for example as described in PCT Application No. PCT/US2018/014111, filed Jan. 17, 2018, and PCT Application No. PCT/US2018/049259, filed Aug. 31, 2018, the entirety of each of which is incorporated herein by reference, or commercially available as ONCOMINE™ TCR Beta-SR Assay DNA, ONCOMINE™ TCR Beta-SR Assay RNA, and ONCOMINE™ TCR Beta-LR Assay (Thermo Fisher Scientific). The ability to assess both the BCR (e.g., IgH) and TCR (e.g., TCR beta) repertoires from a sample using a single multiplex amplification reaction is useful in saving time and limited biological sample and is applicable in many of the methods described herein, including methods related to allergy and autoimmunity, vaccine development and use, and immune-oncology. For example, combining B cell repertoire analysis with T cell repertoire analysis may be used to improve detection of changes in the immune repertoire following administration of immunotherapy, such as checkpoint blockade or checkpoint inhibitor immunotherapy, potentially indicating a response to the immunotherapy. Also, combining B cell repertoire analysis with T cell repertoire analysis may be used to improve evaluation of vaccine efficacy. Exemplary immune repertoire changes in response to immunotherapy or in response to vaccine administration include, without limitation, a decrease in T and B cell evenness following treatment (for example without limitation, at day 7-14 post treatment) in comparison to the pretreatment evenness values, and an increase in the representation of IgG1 expressing B cells following treatment(s) in comparison to the pretreatment values.

In some embodiments, methods and compositions are provided for identifying and/or characterizing immune repertoire clonal populations in a sample from a subject, comprising performing one or more multiplex amplification reactions with the sample or with cDNA prepared from the sample to amplify immune repertoire nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of a respective target C gene of the immune receptor coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying one or more immune repertoire clonal populations for the target BCR from the sample. In particular, embodiments determining the sequence of the immune receptor amplicon molecules includes obtaining initial sequence reads, aligning the initial sequence read to a reference sequence and identifying productive reads, correcting one or more indel errors to generate rescued productive sequence reads; and determining the sequences of the resulting immune receptor molecules. In other embodiments of such methods and compositions, the one or more multiplex amplification reaction is performed using at least one set of primers comprising i) a plurality of V gene primers directed to a majority of different V genes of at least one BCR coding sequence comprising at least a portion of framework region 3 (FR3) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK. In other embodiments of such methods and compositions, the one or more multiplex amplification reaction is performed using at least one set of primers comprising i) a plurality of V gene primers directed to a majority of different V genes of at least one BCR coding sequence comprising at least a portion of framework region 2 (FR2) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK.

In some embodiments, methods and compositions are provided for identifying and/or characterizing immune repertoire clonal populations in a sample from a subject, comprising performing one or more multiplex amplification reactions with the sample or with cDNA prepared from the sample to amplify immune repertoire nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying one or more immune repertoire clonal populations for the target BCR from the sample. In particular, embodiments determining the sequence of the immune receptor amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting immune receptor molecules. In other embodiments of such methods and compositions, the multiplex amplification reaction is performed using at least one set of primers comprising i) a plurality of V gene primers directed to a majority of different V genes of at least one BCR coding sequence comprising at least a portion of framework region 1 (FR1) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of framework region 2 (FR2) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK.

In some embodiments, methods, compositions and workflows provided are for use in evaluating clonal evolution. For example, analysis of clonal lineages may reveal isotype switching and IgH residues important for antigen binding. In some embodiments, methods, compositions and workflows provided are for use in evaluating isotype abundance. For example, over or under representation of certain isotypes may indicate disease or immunodeficiency such as, without limitation, elevated IgG1 in response to viral infection, elevated IgE in allergy, and missing or underrepresented isotypes may indicate primary immunodeficiency. In some embodiments, methods, compositions and workflows provided are for use in quantifying somatic hypermutation. For example, the frequency of somatic hypermutation provides insight into the stage of B cell development at which malignant transformation occurred.

In some embodiments, methods and compositions provided are used to identify and/or characterize somatic hypermutations (SEIM) within a BCR repertoire or clonal populations. In some embodiments, methods and compositions provided are used to identify and/or screen for rare BCR clones or subclones, for example those having somatically hypermutated VDJ rearrangements. In some embodiments, identification, quantification and/or characterization of rare BCR clones may provide biomarkers for a given condition or treatment response. Accordingly, in some embodiments, methods and compositions provided herein are used to identify, screen for and/or characterize BCR clones as biomarkers using samples obtained for example from retrospective or longitudinal subject studies.

In some embodiments, methods for identifying and/or characterizing BCR clonal lineages and SHM comprise performing one or more multiplex amplification reaction with a subject's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and one or more C gene primers directed to at least a portion of a respective target C gene of the BCR coding sequence, sequencing the resultant BCR amplicons, and performing VDJ sequence analysis provided herein to identify and/or quantify SMH and clonal lineages for the target BCR from the sample. In other embodiments, methods for identifying and/or characterizing BCR clonal lineages and SHM comprise performing one or more multiplex amplification reaction with a subject's sample to amplify BCR nucleic acid template molecules having a J gene portion and a variable portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, and performing VDJ sequence analysis provided herein to identify SHM and clonal lineages for the target BCR from the sample.

In some embodiments, methods and compositions provided are used for identifying, quantifying, characterizing and/or monitoring isotype (or sub-isotype) class or isotype class switching within a BCR repertoire or B cell clonal lineage. In some embodiments, such methods comprise performing one or more multiplex amplification reaction with a subject's sample to amplify IgH nucleic acid template molecules having a constant portion and a variable portion using at least one set of primers directed to a majority of different IgH V gene coding sequences comprising at least a portion of FR1, FR2 or FR3 within the V gene, and one or more C gene primers directed to at least a portion of a C gene of the IgH coding sequence, sequencing the resultant amplicons, performing sequence analysis provided herein to identify the IgH isotype class(es) of the BCR repertoire or clonal lineages of the sample. In some embodiments, the primer set comprises one or more primers directed to at least a portion of a C gene of a single isotype, e.g., IgE. In other embodiments, the primer set comprises at least two primers each directed to at least a portion of a C gene of two different isotypes. In other embodiments, the primer set comprises at least one primer separately directed to at least a portion of a C gene of IgA, IgD, IgG, IgM and IgE isotype classes.

In some embodiments, the disclosure provides methods for performing target-specific multiplex PCR on a cDNA sample having a plurality of expressed immune receptor target sequences using primers having a cleavable group.

In certain embodiments, library and/or template preparation to be sequenced are prepared automatically from a population of nucleic acid samples using the compositions provided herein using an automated systems, e.g., the ION CHEF™ system.

As used herein, the term “subject” includes a person, a patient, an individual, someone being evaluated, etc.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or.

As used herein, “antigen” refers to any substance that, when introduced into a body, e.g., of a subject, can stimulate an immune response, such as the production of an antibody or T cell receptor that recognizes the antigen. Antigens include molecules such as nucleic acids, lipids, ribonucleoprotein complexes, protein complexes, proteins, polypeptides, peptides and naturally occurring or synthetic modifications of such molecules against which an immune response involving T and/or B lymphocytes can be generated. With regard to autoimmune disease, the antigens herein are often referred to as autoantigens. With regard to allergic disease the antigens herein are often referred to as allergens. Autoantigens are any molecule produced by the organism that can be the target of an immunologic response, including peptides, polypeptides, and proteins encoded within the genome of the organism and post-translationally-generated modifications of these peptides, polypeptides, and proteins. Such molecules also include carbohydrates, lipids and other molecules produced by the organism. Antigens also include vaccine antigens, which include, without limitation, pathogen antigens, cancer associated antigens, allergens, and the like.

As used herein, “amplify”, “amplifying” or “amplification reaction” and their derivatives, refer to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes PCR.

As used herein, “amplification conditions” and its derivatives, refers to conditions suitable for amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences includes PCR conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg²⁺ or Mn²⁺ (e.g., MgCl₂, etc.) and can also include various modifiers of ionic strength.

As used herein, “target sequence” or “target sequence of interest” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As defined herein, “sample” and its derivatives, is used in its broadest sense and includes any specimen, culture and the like that is suspected of including a target. In some embodiments, the sample comprises cDNA, RNA, PNA, LNA, chimeric, hybrid, or multiplex-forms of nucleic acids. The sample can include any biological, clinical, surgical, agricultural, atmospheric or aquatic-based specimen containing one or more nucleic acids. The term also includes any isolated nucleic acid sample such as expressed RNA, fresh-frozen or formalin-fixed paraffin-embedded nucleic acid specimen.

As used herein, “contacting” and its derivatives, when used in reference to two or more components, refers to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution. Where one or more of the referenced components to be contacted includes a plurality (e.g., “contacting a target sequence with a plurality of target-specific primers and a polymerase”), then each member of the plurality can be viewed as an individual component of the contacting process, such that the contacting can include contacting of any one or more members of the plurality with any other member of the plurality and/or with any other referenced component (e.g., some but not all of the plurality of target specific primers can be contacted with a target sequence, then a polymerase, and then with other members of the plurality of target-specific primers) in any order or combination.

As used herein, the term “primer” and its derivatives refer to any polynucleotide that can hybridize to a target sequence of interest. In some embodiments, the primer can also serve to prime nucleic acid synthesis. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer may be comprised of any combination of nucleotides or analogs thereof, which may be optionally linked to form a linear polymer of any suitable length. In some embodiments, the primer is a single-stranded oligonucleotide or polynucleotide. (For purposes of this disclosure, the terms ‘polynucleotide” and “oligonucleotide” are used interchangeably herein and do not necessarily indicate any difference in length between the two). In some embodiments, the primer is single-stranded but it can also be double-stranded. The primer optionally occurs naturally, as in a purified restriction digest, or can be produced synthetically. In some embodiments, the primer acts as a point of initiation for amplification or synthesis when exposed to amplification or synthesis conditions; such amplification or synthesis can occur in a template-dependent fashion and optionally results in formation of a primer extension product that is complementary to at least a portion of the target sequence. Exemplary amplification or synthesis conditions can include contacting the primer with a polynucleotide template (e.g., a template including a target sequence), nucleotides and an inducing agent such as a polymerase at a suitable temperature and pH to induce polymerization of nucleotides onto an end of the target-specific primer. If double-stranded, the primer can optionally be treated to separate its strands before being used to prepare primer extension products. In some embodiments, the primer is an oligodeoxyribonucleotide or an oligoribonucleotide. In some embodiments, the primer can include one or more nucleotide analogs. The exact length and/or composition, including sequence, of the target-specific primer can influence many properties, including melting temperature (Tm), GC content, formation of secondary structures, repeat nucleotide motifs, length of predicted primer extension products, extent of coverage across a nucleic acid molecule of interest, number of primers present in a single amplification or synthesis reaction, presence of nucleotide analogs or modified nucleotides within the primers, and the like. In some embodiments, a primer can be paired with a compatible primer within an amplification or synthesis reaction to form a primer pair consisting or a forward primer and a reverse primer. In some embodiments, the forward primer of the primer pair includes a sequence that is substantially complementary to at least a portion of a strand of a nucleic acid molecule, and the reverse primer of the primer of the primer pair includes a sequence that is substantially identical to at least of portion of the strand. In some embodiments, the forward primer and the reverse primer are capable of hybridizing to opposite strands of a nucleic acid duplex. Optionally, the forward primer primes synthesis of a first nucleic acid strand, and the reverse primer primes synthesis of a second nucleic acid strand, wherein the first and second strands are substantially complementary to each other, or can hybridize to form a double-stranded nucleic acid molecule. In some embodiments, one end of an amplification or synthesis product is defined by the forward primer and the other end of the amplification or synthesis product is defined by the reverse primer. In some embodiments, where the amplification or synthesis of lengthy primer extension products is required, such as amplifying an exon, coding region, or gene, several primer pairs can be created than span the desired length to enable sufficient amplification of the region. In some embodiments, a primer can include one or more cleavable groups. In some embodiments, primer lengths are in the range of about 10 to about 60 nucleotides, about 12 to about 50 nucleotides and about 15 to about 40 nucleotides in length. Typically, a primer is capable of hybridizing to a corresponding target sequence and undergoing primer extension when exposed to amplification conditions in the presence of dNTPs and a polymerase. In some embodiments, the primer includes one or more cleavable groups at one or more locations within the primer.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer is at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that are used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification is performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer is substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

As used herein, “polymerase” and its derivatives, refers to any enzyme that can catalyze the polymerization of nucleotides (including analogs thereof) into a nucleic acid strand. Typically, but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase is a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase is optionally reactivated, for example through the use of heat, chemicals or re-addition of new amounts of polymerase into a reaction mixture. In some embodiments, the polymerase can include a hot-start polymerase or an aptamer based polymerase that optionally is reactivated.

As used herein, the term “nucleotide” and its variants comprise any compound, including without limitation any naturally occurring nucleotide or analog thereof, which can bind selectively to, or is polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. In some embodiments, the nucleotide can optionally include a chain of phosphorus atoms comprising three, four, five, six, seven, eight, nine, ten or more phosphorus atoms. In some embodiments, the phosphorus chain is attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain is linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain has side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in U.S. Pat. No. 7,405,281. In some embodiments, the nucleotide comprises a label and referred to herein as a “labeled nucleotide”; the label of the labeled nucleotide is referred to herein as a “nucleotide label.” In some embodiments, the label is in the form of a fluorescent dye attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. alpha-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

The term “portion” and its variants, as used herein, when used in reference to a given nucleic acid molecule, for example a primer or a template nucleic acid molecule, comprises any number of contiguous nucleotides within the length of the nucleic acid molecule, including the partial or entire length of the nucleic acid molecule.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “complementary” and “complement” and their variants, as used herein, refer to any two or more nucleic acid sequences (e.g., portions or entireties of template nucleic acid molecules, target sequences and/or primers) that can undergo cumulative base pairing at two or more individual corresponding positions in antiparallel orientation, as in a hybridized duplex. Such base pairing can proceed according to any set of established rules, for example according to Watson-Crick base pairing rules or according to some other base pairing paradigm. Optionally there can be “complete” or “total” complementarity between a first and second nucleic acid sequence where each nucleotide in the first nucleic acid sequence can undergo a stabilizing base pairing interaction with a nucleotide in the corresponding antiparallel position on the second nucleic acid sequence. “Partial” complementarity describes nucleic acid sequences in which at least 20%, but less than 100%, of the residues of one nucleic acid sequence are complementary to residues in the other nucleic acid sequence. In some embodiments, at least 50%, but less than 100%, of the residues of one nucleic acid sequence are complementary to residues in the other nucleic acid sequence. In some embodiments, at least 70%, 80%, 90%, 95% or 98%, but less than 100%, of the residues of one nucleic acid sequence are complementary to residues in the other nucleic acid sequence. Sequences are said to be “substantially complementary” when at least 85% of the residues of one nucleic acid sequence are complementary to residues in the other nucleic acid sequence. In some embodiments, two complementary or substantially complementary sequences are capable of hybridizing to each other under standard or stringent hybridization conditions. “Non-complementary” describes nucleic acid sequences in which less than 20% of the residues of one nucleic acid sequence are complementary to residues in the other nucleic acid sequence. Sequences are said to be “substantially non-complementary” when less than 15% of the residues of one nucleic acid sequence are complementary to residues in the other nucleic acid sequence. In some embodiments, two non-complementary or substantially non-complementary sequences cannot hybridize to each other under standard or stringent hybridization conditions. A “mismatch” is present at any position in the sequences where two opposed nucleotides are not complementary. Complementary nucleotides include nucleotides that are efficiently incorporated by DNA polymerases opposite each other during DNA replication under physiological conditions. In a typical embodiment, complementary nucleotides can form base pairs with each other, such as the A-T/U and G-C base pairs formed through specific Watson-Crick type hydrogen bonding, or base pairs formed through some other type of base pairing paradigm, between the nucleobases of nucleotides and/or polynucleotides in positions antiparallel to each other. The complementarity of other artificial base pairs can be based on other types of hydrogen bonding and/or hydrophobicity of bases and/or shape complementarity between bases.

As used herein, “amplified target sequences” and its derivatives, refers to a nucleic acid sequence produced by the amplification of/amplifying the target sequences using target-specific primers and the methods provided herein. The amplified target sequences may be either of the same sense (the positive strand produced in the second round and subsequent even-numbered rounds of amplification) or antisense (i.e., the negative strand produced during the first and subsequent odd-numbered rounds of amplification) with respect to the target sequences. In some embodiments, the amplified target sequences are less than 50% complementary to any portion of another amplified target sequence in the reaction. In other embodiments, the amplified target sequences are greater than 50%, greater than 60%, greater than 70%, greater than 80%, or greater than 90% complementary to any portion of another amplified target sequence in the reaction.

As used herein, the terms “ligating”, “ligation” and their derivatives refer to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the ligation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase is used. For the purposes of this disclosure, an amplified target sequence can be ligated to an adapter to generate an adapter-ligated amplified target sequence.

As used herein, “ligase” and its derivatives, refers to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. In some embodiments, the ligase is an isothermal ligase. In some embodiments, the ligase is a thermostable ligase. Suitable ligases may include, but not limited to, T4 DNA ligase, T4 RNA ligase, and E. coli DNA ligase.

As used herein, “ligation conditions” and its derivatives, refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap is ligated in the presence of an enzyme, such as ligase at an appropriate temperature and pH. In some embodiments, T4 DNA ligase can join a nick between nucleic acids at a temperature of about 70-72° C.

As used herein, “blunt-end ligation” and its derivatives, refers to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang”. In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in US Pat. Publication No. 2010/0129874. In some embodiments, blunt-ended ligation includes a nick translation reaction to seal a nick created during the ligation process.

As used herein, the terms “adapter” or “adapter and its complements” and their derivatives, refers to any linear oligonucleotide which is ligated to a nucleic acid molecule of the disclosure. Optionally, the adapter includes a nucleic acid sequence that is not substantially complementary to the 3′ end or the 5′ end of at least one target sequences within the sample. In some embodiments, the adapter is substantially non-complementary to the 3′ end or the 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. An adapter can include any combination of nucleotides and/or nucleic acids. In some embodiments, the adapter can include one or more cleavable groups at one or more locations. In another embodiment, the adapter can include a sequence that is substantially identical, or substantially complementary, to at least a portion of a primer, for example a universal primer. The structure and properties of universal amplification primers are well known to those skilled in the art and can be implemented for utilization in conjunction with provided methods and compositions to adapt to specific analysis platforms (e.g., as described herein universal P1 and A primers have been described in the art and utilized for sequencing on ION TORRENT™ sequencing platforms). Similarly, additional and other universal adaptor/primer sequences described and known in the art (e.g., Illumina universal adaptor/primer sequences, PacBio universal adaptor/primer sequences, etc.) can be used in conjunction with the methods and compositions provided herein. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

In some embodiments, an adapter is ligated to a polynucleotide through a blunt-end ligation. In other embodiments, an adapter is ligated to a polynucleotide via nucleotide overhangs on the ends of the adapter and the polynucleotide. For overhang ligation, an adapter may have a nucleotide overhang added to the 3′ and/or 5′ ends of the respective strands if the polynucleotides to which the adapters are to be ligated (e.g., amplicons) have a complementary overhang added to the 3′ and/or 5′ ends of the respective strands. For example, adenine nucleotides can be added to the 3′ terminus of an end-repaired PCR product. Adapters having with an overhang formed by thymine nucleotides can then dock with the A-overhang of the amplicon and be ligated to the amplicon by a DNA ligase, such as T4 DNA ligase.

As used herein, “reamplifying” or “reamplification” and their derivatives refer to any process whereby at least a portion of an amplified nucleic acid molecule is further amplified via any suitable amplification process (referred to in some embodiments as a “secondary” amplification or “reamplification”, thereby producing a reamplified nucleic acid molecule. The secondary amplification need not be identical to the original amplification process whereby the amplified nucleic acid molecule was produced; nor need the reamplified nucleic acid molecule be completely identical or completely complementary to the amplified nucleic acid molecule; all that is required is that the reamplified nucleic acid molecule include at least a portion of the amplified nucleic acid molecule or its complement. For example, the reamplification can involve the use of different amplification conditions and/or different primers, including different target-specific primers than the primary amplification.

As defined herein, a “cleavable group” refers to any moiety that once incorporated into a nucleic acid can be cleaved under appropriate conditions. For example, a cleavable group can be incorporated into a target-specific primer, an amplified sequence, an adapter or a nucleic acid molecule of the sample. In an exemplary embodiment, a target-specific primer can include a cleavable group that becomes incorporated into the amplified product and is subsequently cleaved after amplification, thereby removing a portion, or all, of the target-specific primer from the amplified product. The cleavable group can be cleaved or otherwise removed from a target-specific primer, an amplified sequence, an adapter or a nucleic acid molecule of the sample by any acceptable means. For example, a cleavable group can be removed from a target-specific primer, an amplified sequence, an adapter or a nucleic acid molecule of the sample by enzymatic, thermal, photo-oxidative or chemical treatment. In one embodiment, a cleavable group can include a nucleobase that is not naturally occurring. For example, an oligodeoxyribonucleotide can include one or more RNA nucleobases, such as uracil that can be removed by a uracil glycosylase. In some embodiments, a cleavable group can include one or more modified nucleobases (such as 7-methylguanine, 8-oxo-guanine, xanthine, hypoxanthine, 5,6-dihydrouracil or 5-methylcytosine) or one or more modified nucleosides (i.e., 7-methylguanosine, 8-oxo-deoxyguanosine, xanthosine, inosine, dihydrouridine or 5-methylcytidine). The modified nucleobases or nucleotides can be removed from the nucleic acid by enzymatic, chemical or thermal means. In one embodiment, a cleavable group can include a moiety that can be removed from a primer after amplification (or synthesis) upon exposure to ultraviolet light (i.e., bromodeoxyuridine). In another embodiment, a cleavable group can include methylated cytosine. Typically, methylated cytosine can be cleaved from a primer for example, after induction of amplification (or synthesis), upon sodium bisulfite treatment. In some embodiments, a cleavable moiety can include a restriction site. For example, a primer or target sequence can include a nucleic acid sequence that is specific to one or more restriction enzymes, and following amplification (or synthesis), the primer or target sequence can be treated with the one or more restriction enzymes such that the cleavable group is removed. Typically, one or more cleavable groups can be included at one or more locations with a target-specific primer, an amplified sequence, an adapter or a nucleic acid molecule of the sample.

As used herein, “cleavage step” and its derivatives, refers to any process by which a cleavable group is cleaved or otherwise removed from a target-specific primer, an amplified sequence, an adapter or a nucleic acid molecule of the sample. In some embodiments, the cleavage step involves a chemical, thermal, photo-oxidative or digestive process.

As used herein, the term “hybridization” is consistent with its use in the art, and refers to the process whereby two nucleic acid molecules undergo base pairing interactions. Two nucleic acid molecule molecules are said to be hybridized when any portion of one nucleic acid molecule is base paired with any portion of the other nucleic acid molecule; it is not necessarily required that the two nucleic acid molecules be hybridized across their entire respective lengths and in some embodiments, at least one of the nucleic acid molecules can include portions that are not hybridized to the other nucleic acid molecule. The phrase “hybridizing under stringent conditions” and its variants refers to conditions under which hybridization of a target-specific primer to a target sequence occurs in the presence of high hybridization temperature and low ionic strength. In one exemplary embodiment, stringent hybridization conditions include an aqueous environment containing about 30 mM magnesium sulfate, about 300 mM Tris-sulfate at pH 8.9, and about 90 mM ammonium sulfate at about 60-68° C., or equivalents thereof. As used herein, the phrase “standard hybridization conditions” and its variants refers to conditions under which hybridization of a primer to an oligonucleotide (i.e., a target sequence), occurs in the presence of low hybridization temperature and high ionic strength. In one exemplary embodiment, standard hybridization conditions include an aqueous environment containing about 100 mM magnesium sulfate, about 500 mM Tris-sulfate at pH 8.9, and about 200 mM ammonium sulfate at about 50-55° C., or equivalents thereof.

As used herein, “GC content” and its derivatives, refers to the cytosine and guanine content of a nucleic acid molecule. The GC content of a target-specific primer (or adapter) of the disclosure is 85% or lower. More typically, the GC content of a target-specific primer or adapter of the disclosure is between 15-85%.

As used herein, the term “end” and its variants, when used in reference to a nucleic acid molecule, for example a target sequence or amplified target sequence, can include the terminal 30 nucleotides, the terminal 20 and even more typically the terminal 15 nucleotides of the nucleic acid molecule. A linear nucleic acid molecule comprised of linked series of contiguous nucleotides typically includes at least two ends. In some embodiments, one end of the nucleic acid molecule can include a 3′ hydroxyl group or its equivalent, and is referred to as the “3′ end” and its derivatives. Optionally, the 3′ end includes a 3′ hydroxyl group that is not linked to a 5′ phosphate group of a mononucleotide pentose ring. Typically, the 3′ end includes one or more 5′ linked nucleotides located adjacent to the nucleotide including the unlinked 3′ hydroxyl group, typically the 30 nucleotides located adjacent to the 3′ hydroxyl, typically the terminal 20 and even more typically the terminal 15 nucleotides. One or more linked nucleotides can be represented as a percentage of the nucleotides present in the oligonucleotide or can be provided as a number of linked nucleotides adjacent to the unlinked 3′ hydroxyl. For example, the 3′ end can include less than 50% of the nucleotide length of the oligonucleotide. In some embodiments, the 3′ end does not include any unlinked 3′ hydroxyl group but can include any moiety capable of serving as a site for attachment of nucleotides via primer extension and/or nucleotide polymerization. In some embodiments, the term “3′ end” for example when referring to a target-specific primer, can include the terminal 10 nucleotides, the terminal 5 nucleotides, the terminal 4, 3, 2 or fewer nucleotides at the 3′end. In some embodiments, the term “3′ end” when referring to a target-specific primer can include nucleotides located at nucleotide positions 10 or fewer from the 3′ terminus.

As used herein, “5′ end”, and its derivatives, refers to an end of a nucleic acid molecule, for example a target sequence or amplified target sequence, which includes a free 5′ phosphate group or its equivalent. In some embodiments, the 5′ end includes a 5′ phosphate group that is not linked to a 3′ hydroxyl of a neighboring mononucleotide pentose ring. Typically, the 5′ end includes to one or more linked nucleotides located adjacent to the 5′ phosphate, typically the 30 nucleotides located adjacent to the nucleotide including the 5′ phosphate group, typically the terminal 20 and even more typically the terminal 15 nucleotides. One or more linked nucleotides can be represented as a percentage of the nucleotides present in the oligonucleotide or can be provided as a number of linked nucleotides adjacent to the 5′ phosphate. For example, the 5′ end can be less than 50% of the nucleotide length of an oligonucleotide. In another exemplary embodiment, the 5′ end can include about 15 nucleotides adjacent to the nucleotide including the terminal 5′ phosphate. In some embodiments, the 5′ end does not include any unlinked 5′ phosphate group but can include any moiety capable of serving as a site of attachment to a 3′ hydroxyl group, or to the 3′end of another nucleic acid molecule. In some embodiments, the term “5′ end” for example when referring to a target-specific primer, can include the terminal 10 nucleotides, the terminal 5 nucleotides, the terminal 4, 3, 2 or fewer nucleotides at the 5′end. In some embodiments, the term “5′ end” when referring to a target-specific primer can include nucleotides located at positions 10 or fewer from the 5′ terminus. In some embodiments, the 5′ end of a target-specific primer can include only non-cleavable nucleotides, for example nucleotides that do not contain one or more cleavable groups as disclosed herein, or a cleavable nucleotide as would be readily determined by one of ordinary skill in the art.

As used herein, “DNA barcode” and its derivatives, refers to a unique short (e.g., 6-14 nucleotide) nucleic acid sequence within an adapter that can act as a ‘key’ to distinguish or separate a plurality of amplified target sequences in a sample. For the purposes of this disclosure, a DNA barcode can be incorporated into the nucleotide sequence of an adapter.

As used herein, the phrases “two rounds of target-specific hybridization” or “two rounds of target-specific selection” and their derivatives refers to any process whereby the same target sequence is subjected to two consecutive rounds of hybridization-based target-specific selection, wherein a target sequence is hybridized to a target-specific sequence. Each round of hybridization based target-specific selection can include multiple target-specific hybridizations to at least some portion of a target-specific sequence. In one exemplary embodiment, a round of target-specific selection includes a first target-specific hybridization involving a first region of the target sequence and a second target-specific hybridization involving a second region of the target sequence. The first and second regions can be the same or different. In some embodiments, each round of hybridization-based target-specific selection can include use of two target specific oligonucleotides (e.g., a forward target-specific primer and a reverse target-specific primer), such that each round of selection includes two target-specific hybridizations.

As used herein, “comparable maximal minimum melting temperatures” and its derivatives, refers to the melting temperature (Tₘ) of each nucleic acid fragment for a single adapter or target-specific primer after cleavage of the cleavable groups. The hybridization temperature of each nucleic acid fragment generated by a single adapter or target-specific primer is compared to determine the maximal minimum temperature required preventing hybridization of any nucleic acid fragment from the target-specific primer or adapter to the target sequence. Once the maximal hybridization temperature is known, it is possible to manipulate the adapter or target-specific primer, for example by moving the location of the cleavable group along the length of the primer, to achieve a comparable maximal minimum melting temperature with respect to each nucleic acid fragment.

As used herein, “addition only” and its derivatives, refers to a series of steps in which reagents and components are added to a first or single reaction mixture. Typically, the series of steps excludes the removal of the reaction mixture from a first vessel to a second vessel in order to complete the series of steps. An addition only process excludes the manipulation of the reaction mixture outside the vessel containing the reaction mixture. Typically, an addition-only process is amenable to automation and high-throughput.

As used herein, “synthesizing” and its derivatives, refers to a reaction involving nucleotide polymerization by a polymerase, optionally in a template-dependent fashion. Polymerases synthesize an oligonucleotide via transfer of a nucleoside monophosphate from a nucleoside triphosphate (NTP), deoxynucleoside triphosphate (dNTP) or dideoxynucleoside triphosphate (ddNTP) to the 3′ hydroxyl of an extending oligonucleotide chain. For the purposes of this disclosure, synthesizing includes to the serial extension of a hybridized adapter or a target-specific primer via transfer of a nucleoside monophosphate from a deoxynucleoside triphosphate.

As used herein, “polymerizing conditions” and its derivatives, refers to conditions suitable for nucleotide polymerization. In typical embodiments, such nucleotide polymerization is catalyzed by a polymerase. In some embodiments, polymerizing conditions include conditions for primer extension, optionally in a template-dependent manner, resulting in the generation of a synthesized nucleic acid sequence. In some embodiments, the polymerizing conditions include PCR. Typically, the polymerizing conditions include use of a reaction mixture that is sufficient to synthesize nucleic acids and includes a polymerase and nucleotides. The polymerizing conditions can include conditions for annealing of a target-specific primer to a target sequence and extension of the primer in a template dependent manner in the presence of a polymerase. In some embodiments, polymerizing conditions are practiced using thermocycling. Additionally, polymerizing conditions can include a plurality of cycles where the steps of annealing, extending, and separating the two nucleic strands are repeated. Typically, the polymerizing conditions include a cation such as MgCl₂. Polymerization of one or more nucleotides to form a nucleic acid strand includes that the nucleotides be linked to each other via phosphodiester bonds, however, alternative linkages may be possible in the context of particular nucleotide analogs.

As used herein, the term “nucleic acid” refers to natural nucleic acids, artificial nucleic acids, analogs thereof, or combinations thereof, including polynucleotides and oligonucleotides. As used herein, the terms “polynucleotide” and “oligonucleotide” are used interchangeably and mean single-stranded and double-stranded polymers of nucleotides including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH⁴⁺, trialkylammonium, Mg²⁺, Na⁺ and the like. An oligonucleotide can be composed entirely of deoxyribonucleotides, entirely of ribonucleotides, or chimeric mixtures thereof. Oligonucleotides can be comprised of nucleobase and sugar analogs. Polynucleotides typically range in size from a few monomeric units, e.g. 5-40, when they are more commonly frequently referred to in the art as oligonucleotides, to several thousands of monomeric nucleotide units, when they are more commonly referred to in the art as polynucleotides; for purposes of this disclosure, however, both oligonucleotides and polynucleotides may be of any suitable length. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U′ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

As defined herein, the term “nick translation” and its variants comprise the translocation of one or more nicks or gaps within a nucleic acid strand to a new position along the nucleic acid strand. In some embodiments, a nick is formed when a double stranded adapter is ligated to a double stranded amplified target sequence. In one example, the primer can include at its 5′ end, a phosphate group that can ligate to the double stranded amplified target sequence, leaving a nick between the adapter and the amplified target sequence in the complementary strand. In some embodiments, nick translation results in the movement of the nick to the 3′ end of the nucleic acid strand. In some embodiments, moving the nick can include performing a nick translation reaction on the adapter-ligated amplified target sequence. In some embodiments, the nick translation reaction is a coupled 5′ to 3′ DNA polymerization/degradation reaction, or coupled to a 5′ to 3′ DNA polymerization/strand displacement reaction. In some embodiments, moving the nick can include performing a DNA strand extension reaction at the nick site. In some embodiments, moving the nick can include performing a single strand exonuclease reaction on the nick to form a single stranded portion of the adapter-ligated amplified target sequence and performing a DNA strand extension reaction on the single stranded portion of the adapter-ligated amplified target sequence to a new position. In some embodiments, a nick is formed in the nucleic acid strand opposite the site of ligation.

As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a polynucleotide of interest in a mixture of expressed RNA or cDNA without cloning or purification. This process for amplifying the polynucleotide of interest consists of introducing a large excess of two oligonucleotide primers to the DNA mixture containing the desired polynucleotide of interest, followed by a precise sequence of thermal cycling in the presence of a DNA polymerase. The two primers are complementary to their respective strands of the double stranded polynucleotide of interest. To effect amplification, the mixture is denatured and the primers then annealed to their complementary sequences within the polynucleotide of interest molecule. Following annealing, the primers are extended with a polymerase to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “PCR”. Because the desired amplified segments of the polynucleotide of interest become the predominant nucleic acid sequences (in terms of concentration) in the mixture, they are said to be “PCR amplified”. As defined herein, target nucleic acid molecules within a sample including a plurality of target nucleic acid molecules are amplified via PCR. In a modification to the method discussed above, the target nucleic acid molecules are PCR amplified using a plurality of different primer pairs, in some cases, one or more primer pairs per target nucleic acid molecule of interest, thereby forming a multiplex PCR reaction. In some embodiments provided herein, multiplex PCR amplifications are performed using a plurality of different primer pairs, in typical cases, one primer pair per target nucleic acid molecule. Using multiplex PCR, it is possible to simultaneously amplify multiple nucleic acid molecules of interest from a sample to form amplified target sequences. It is also possible to detect the amplified target sequences by several different methodologies (e.g., quantitation with a bioanalyzer or qPCR, hybridization with a labeled probe; incorporation of biotinylated primers followed by avidin-enzyme conjugate detection; incorporation of ³²P-labeled deoxynucleotide triphosphates, such as dCTP or dATP, into the amplified target sequence). Any oligonucleotide sequence can be amplified with the appropriate set of primers, thereby allowing for the amplification of target nucleic acid molecules from RNA, cDNA, formalin-fixed paraffin-embedded DNA, fine-needle biopsies and various other sources. In particular, the amplified target sequences created by the multiplex PCR process as disclosed herein, are themselves efficient substrates for subsequent PCR amplification or various downstream assays or manipulations.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers to the number of different target-specific sequences that are amplified during that single multiplex amplification. In some embodiments, the plexy is about 12-plex, 24-plex, 48-plex, 74-plex, 96-plex, 120-plex, 144-plex, 168-plex, 192-plex, 216-plex, 240-plex, 264-plex, 288-plex, 312-plex, 336-plex, 360-plex, 384-plex, or 398-plex. In some embodiments, highly multiplexed amplification reactions include reactions with a plexy of greater than 12-plex.

In some embodiments, the amplified target sequences are formed via PCR. Extension of target-specific primers can be accomplished using one or more DNA polymerases. In one embodiment, the polymerase is any Family A DNA polymerase (also known as pol I family) or any Family B DNA polymerase. In some embodiments, the DNA polymerase is a recombinant form capable of extending target-specific primers with superior accuracy and yield as compared to a non-recombinant DNA polymerase. For example, the polymerase can include a high-fidelity polymerase or thermostable polymerase. In some embodiments, conditions for extension of target-specific primers can include ‘Hot Start’ conditions, for example Hot Start polymerases, such as AMPLITAQ GOLD® DNA polymerase (Applied Biosciences), PLATINUM® Taq DNA Polymerase High Fidelity (Invitrogen) or KOD Hot Start DNA polymerase (EMD Biosciences). A ‘Hot Start’ polymerase includes a thermostable polymerase and one or more antibodies that inhibit DNA polymerase and 3′-5′ exonuclease activities at ambient temperature. In some instances, ‘Hot Start’ conditions can include an aptamer.

In some embodiments, the polymerase is an enzyme such as Taq polymerase (from Thermus aquaticus), Tfi polymerase (from Thermus filiformis), Bst polymerase (from Bacillus stearothermophilus), Pfu polymerase (from Pyrococcus furiosus), Tth polymerase (from Thermus thermophilus), Pow polymerase (from Pyrococcus woesei), Tli polymerase (from Thermococcus litoralis), Ultima polymerase (from Thermotoga maritima), KOD polymerase (from Thermococcus kodakaraensis), Pol I and II polymerases (from Pyrococcus abyssi) and Pab (from Pyrococcus abyssi). In some embodiments, the DNA polymerase can include at least one polymerase such as AMPLITAQ GOLD® DNA polymerase (Applied Biosciences), Stoffel fragment of AMPLITAQ® DNA Polymerase (Roche), KOD polymerase (EMD Biosciences), KOD Hot Start polymerase (EMD Biosciences), DEEP VENT™ DNA polymerase (New England Biolabs), Phusion polymerase (New England Biolabs), Klentaq1 polymerase (DNA Polymerase Technology, Inc), Klentaq Long Accuracy polymerase (DNA Polymerase Technology, Inc), OMNI KLENTAQ™ DNA polymerase (DNA Polymerase Technology, Inc), OMNI KLENTAQ™ LA DNA polymerase (DNA Polymerase Technology, Inc), PLATINUM® Taq DNA Polymerase (Invitrogen), HEMO KLENTAQ™ (New England Biolabs), PLATINUM® Taq DNA Polymerase High Fidelity (Invitrogen), PLATINUM® Pfx (Invitrogen), ACCUPRIME™ Pfx (Invitrogen), or ACCUPRIME™ Taq DNA Polymerase High Fidelity (Invitrogen).

In some embodiments, the DNA polymerase is a thermostable DNA polymerase. In some embodiments, the mixture of dNTPs is applied concurrently, or sequentially, in a random or defined order. In some embodiments, the amount of DNA polymerase present in the multiplex reaction is significantly higher than the amount of DNA polymerase used in a corresponding single plex PCR reaction. As defined herein, the term “significantly higher” refers to an at least 3-fold greater concentration of DNA polymerase present in the multiplex PCR reaction as compared to a corresponding single plex PCR reaction.

In some embodiments, the amplification reaction does not include a circularization of amplification product, for example as disclosed by rolling circle amplification.

The practice of the present subject matter may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, polymerization techniques, chemical and physical analysis of polymer particles, preparation of nucleic acid libraries, nucleic acid sequencing and analysis, and the like. Specific illustrations of suitable techniques can be used by reference to the examples provided herein. Other equivalent conventional procedures can also be used. Such conventional techniques and descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press), Hermanson, Bioconjugate Techniques, Second Edition (Academic Press, 2008); Merkus, Particle Size Measurements (Springer, 2009); Rubinstein and Colby, Polymer Physics (Oxford University Press, 2003); and the like.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

Various additional exemplary embodiments may be derived by repeating, adding, or substituting any generically or specifically described features and/or components and/or substances and/or steps and/or operating conditions set forth in one or more of the above-described exemplary embodiments. Further, it should be understood that an order of steps or order for performing certain actions is immaterial so long as the objective of the steps or action remains achievable, unless specifically stated otherwise. Furthermore, two or more steps or actions can be conducted simultaneously so long as the objective of the steps or action remains achievable, unless specifically stated otherwise. Moreover, any one or more feature, component, aspect, step, or other characteristic mentioned in one of the above-discussed exemplary embodiments may be considered to be a potential optional feature, component, aspect, step, or other characteristic of any other of the above-discussed exemplary embodiments so long as the objective of such any other of the above-discussed exemplary embodiments remains achievable, unless specifically stated otherwise.

In certain embodiments, compositions comprise target BCR primer sets wherein the primers are directed to sequences of the same target BCR gene. In some embodiments the immune receptor is an antibody receptor selected from the group consisting of heavy chain alpha, heavy chain delta, heavy chain epsilon, heavy chain gamma, heavy chain mu, light chain kappa, and light chain lambda. In some embodiments, a target BCR primer set can be combined with a primer set directed to a TCR selected from the group consisting of TCR alpha, TCR beta, TCR gamma, and TCR delta.

In some embodiments, the amplicon library prepared using target—specific primer pairs can be used in downstream enrichment applications such as emulsion PCR, bridge PCR or isothermal amplification. In some embodiments, the amplicon library can be used in an enrichment application and a sequencing application. For example, an amplicon library can be sequenced using any suitable DNA sequencing platform, including any suitable next generation DNA sequencing platform. In some embodiments, an amplicon library can be sequenced using an ION PGM™ Sequencer or an ION GENESTUDIO™ S5 Sequencer (Thermo Fisher Scientific). In some embodiments, an ION PGM™ Sequencer (Thermo Fisher Scientific) or an S5 Sequencer can be coupled to server that applies parameters or software to determine the sequence of the amplified target nucleic acid molecules. In some embodiments, the amplicon library can be prepared, enriched and sequenced in less than 24 hours. In some embodiments, the amplicon library can be prepared, enriched and sequenced in approximately 9 hours.

In some embodiments, methods for generating an amplicon library can include: amplifying cDNA of immune receptor genes using V gene-specific and C gene-specific primers to generate amplicons; purifying the amplicons from the input DNA and primers; phosphorylating the amplicons; ligating adapters to the phosphorylated amplicons; purifying the ligated amplicons; nick-translating the amplified amplicons; and purifying the nick-translated amplicons to generate the amplicon library. In some embodiments, methods for generating an amplicon library can include: amplifying cDNA of immune receptor genes using V gene-specific and J gene-specific primers to generate amplicons; purifying the amplicons from the input DNA and primers; phosphorylating the amplicons; ligating adapters to the phosphorylated amplicons; purifying the ligated amplicons; nick-translating the amplified amplicons; and purifying the nick-translated amplicons to generate the amplicon library. In some embodiments, additional amplicon library manipulations can be conducted following the step of amplification of rearranged immune receptor gene targets to generate the amplicons. In some embodiments, any combination of additional reactions can be conducted in any order, and can include: purifying; phosphorylating; ligating adapters; nick-translating; amplification and/or sequencing. In some embodiments, any of these reactions can be omitted or can be repeated. It will be readily apparent to one of skill in the art that the method can repeat or omit any one or more of the above steps. It will also be apparent to one of skill in the art that the order and combination of steps may be modified to generate the required amplicon library, and is not therefore limited to the exemplary methods provided.

A phosphorylated amplicon can be joined to an adapter to conduct a nick translation reaction, subsequent downstream amplification (e.g., template preparation), or for attachment to particles (e.g., beads), or both. For example, an adapter that is joined to a phosphorylated amplicon can anneal to an oligonucleotide capture primer which is attached to a particle, and a primer extension reaction can be conducted to generate a complimentary copy of the amplicon attached to the particle or surface, thereby attaching an amplicon to a surface or particle. Adapters can have one or more amplification primer hybridization sites, sequencing primer hybridization sites, barcode sequences, and combinations thereof. In some embodiments, amplicons prepared by the methods disclosed herein can be joined to one or more ION TORRENT™ compatible adapters to construct an amplicon library. Amplicons generated by such methods can be joined to one or more adapters for library construction to be compatible with a next generation sequencing platform. For example, the amplicons produced by the teachings of the present disclosure can be attached to adapters provided in the ION AMPLISEQ™ Library Kit 2.0 or ION AMPLISEQ™ Library Kit Plus (Thermo Fisher Scientific).

In some embodiments, amplification of immune receptor cDNA or rearranged gDNA can be conducted using a 5× ION AIVIPLISEQ™ HiFi Master Mix. In some embodiments, the 5×ION AMPLISEQ™ HiFi Master Mix can include glycerol, dNTPs, and a DNA polymerase such as PLATINUM™ Taq DNA polymerase High Fidelity. In some embodiments, the 5×ION AIVIPLISEQ™ HiFi Master Mix can further include at least one of the following: a preservative, magnesium chloride, magnesium sulfate, tris-sulfate and/or ammonium sulfate.

In some embodiments, the immune receptor rearranged gDNA multiplex amplification reaction further includes at least one PCR additive to improve on-target amplification, amplification yield, and/or the percentage of productive sequencing reads. In some embodiments, the at least one PCR additive includes at least one of potassium chloride or additional dNTPs (e.g., dATP, dCTP, dGTP, dTTP). In some embodiments, the dNTPs as a PCR additive is an equimolar mixture of dNTPs. In some embodiments, the dNTP mix as a PCR additive is an equimolar mixture of dATP, dCTP, dGTP, and dTTP. In some embodiments, about 0.2 mM to about 5.0 mM dNTPs is added to the multiplex amplification reaction. In some embodiments, amplification of rearranged immune receptor gDNA can be conducted using a 5×ION AMPLISEQ™ HiFi Master Mix and an additional about 0.2 mM to about 5.0 mM dNTPs in the reaction mixture. In some embodiments, amplification of rearranged immune receptor gDNA can be conducted using a 5×ION AIVIPLISEQ™ HiFi Master Mix and an additional about 0.5 mM to about 4 mM, about 0.5 mM to about 3 mM, about 0.5 mM to about 2.5 mM, about 0.5 mM to about 1.0 mM, about 0.75 mM to about 1.25 mM, about 1.0 mM to about 1.5 mM, about 1.0 to about 2.0 mM, about 2.0 mM to about 3.0 mM, about 1.25 to about 1.75 mM, about 1.3 to about 1.8 mM, about 1.4 mM to about 1.7 mM, or about 1.5 to about 2.0 mM dNTPs in the reaction mixture. In some embodiments, amplification of rearranged immune receptor gDNA can be conducted using a 5×ION AMPLISEQ™ HiFi Master Mix and an additional about 0.2 mM, about 0.4 mM, about 0.6 mM, about 0.8 mM, about 1.0 mM, about 1.2 mM, about 1.4 mM, about 1.6 mM, about 1.8 mM, about 2.0 mM, about 2.2 mM, about 2.4 mM, about 2.6 mM, about 2.8 mM, about 3.0 mM, about 3.5 mM, or about 4.0 mM dNTPs in the reaction mixture. In some embodiments, about 10 mM to about 200 mM potassium chloride is added to the multiplex amplification reaction. In some embodiments, amplification of rearranged immune receptor gDNA can be conducted using a 5×ION AMPLISEQ™ HiFi Master Mix and an additional about 10 mM to about 200 mM potassium chloride in the reaction mixture. In some embodiments, amplification of rearranged immune receptor gDNA can be conducted using a 5×ION AIVIPLISEQ™ HiFi Master Mix and an additional about 10 mM to about 60 mM, about 20 mM to about 70 mM, about 30 mM to about 80 mM, about 40 mM to about 90 mM, about 50 mM to about 100 mM, about 60 mM to about 120 mM, about 80 mM to about 140 mM, about 50 mM to about 150 mM, about 150 mM to about 200 mM or about 100 mM to about 200 mM potassium chloride in the reaction mixture. In some embodiments, amplification of rearranged immune receptor gDNA can be conducted using a 5×ION AIVIPLISEQ™ HiFi Master Mix and an additional about 10 mM, about 20 mM, about 30 mM, about 40 mM, about 50 mM, about 60 mM, about 70 mM, about 80 mM, about 90 mM, about 100 mM, about 120 mM, about 140 mM, about 150 mM, about 160 mM, about 180 mM, or about 200 mM potassium chloride in the reaction mixture.

In some embodiments, phosphorylation of the amplicons can be conducted using a FuPa reagent. In some embodiments, the FuPa reagent can include a DNA polymerase, a DNA ligase, at least one uracil cleaving or modifying enzyme, and/or a storage buffer. In some embodiments, the FuPa reagent can further include at least one of the following: a preservative and/or a detergent.

In some embodiments, phosphorylation of the amplicons can be conducted using a FuPa reagent. In some embodiments, the FuPa reagent can include a DNA polymerase, at least one uracil cleaving or modifying enzyme, an antibody and/or a storage buffer. In some embodiments, the FuPa reagent can further include at least one of the following: a preservative and/or a detergent. In some embodiments, the antibody is provided to inhibit the DNA polymerase and 3′-5′ exonuclease activities at ambient temperature.

In some embodiments, the amplicon library produced by the teachings of the present disclosure are sufficient in yield to be used in a variety of downstream applications including the ION CHEF™ instrument and the ION S5™ Sequencing Systems (Thermo Fisher Scientific).

It will be apparent to one of ordinary skill in the art that numerous other techniques, platforms or methods for clonal amplification such as wildfire PCR and bridge amplification can be used in conjunction with the amplified target sequences of the present disclosure. It is also envisaged that one of ordinary skill in art upon further refinement or optimization of the conditions provided herein can proceed directly to nucleic acid sequencing (for example using ION PGM™ System or ION S5™ System or ION PROTON™ System sequencers, Thermo Fisher Scientific) without performing a clonal amplification step.

In some embodiments, at least one of the amplified targets sequences to be clonally amplified can be attached to a support or particle. The support can be comprised of any suitable material and have any suitable shape, including, for example, planar, spheroid or particulate. In some embodiments, the support is a scaffolded polymer particle as described in U.S. Published App. No. 20100304982, hereby incorporated by reference in its entirety.

### EXAMPLES

As described above, peripheral blood leukocytes (PBL) were extracted from 99 rheumatoid arthritis patients at baseline (time of first methotrexate administration), month 6 and month 12 post-treatment. A sample of 25 ng of total RNA from PBL was used for targeted AMPLISEQ™ IGH sequencing via the IGH-LR assay with 7 samples per 530 chip on the GENESTUDIO™ S5 (Thermo Fisher Scientific), and sequenced to a target of 1.5M reads per sample. Reads from the sequencer were processed and analyzed via the ION REPORTER′ Software (Thermo Fisher Scientific) using the BCR IGH-LR workflow with settings for bidirectional support required and full length read required set to “true”. The BCH LGH-LR workflow output provided measurements of the features, including IGHM clone frequency, IGHD clone frequency, IGHA1+IGHA2 clone frequency, IGHG3+IGHG4 clone frequency, IGHM highly mutated (>10%) clone frequency and IGHG1 highly mutated (>10%) clone frequency. Sample metadata pertaining to age, gender, clinical disease activity index score (CDAI), smoking status and response information (R: responder or N: non-responder) were collected from the site of collection.

The features, including CDAI, IGHM clone frequency, IGHD clone frequency, IGHA1+IGHA2 clone frequency, IGHG3+IGHG4 clone frequency, IGHM highly mutated (>10%) clone frequency, IGHG1 highly mutated (>10%) clone frequency. The plurality of decision trees, as described with respect to FIGS. 2A-2X, were applied to the features to produce a plurality of output values, as described above. In this example, 76 component decision trees produced 76 output values. The sum of the output values was calculated as SUM=v(1)+v(2)+ . . . v(N), where v(i) is the output value from the i-th decision tree and N=76 for this example. The sigmoid function was applied to the sum to give the prediction value, as described with respect to equation (1). The prediction value was compared to a final threshold of 0.5 to identify the subject as a likely responder or a likely non-responder to methotrexate treatment.

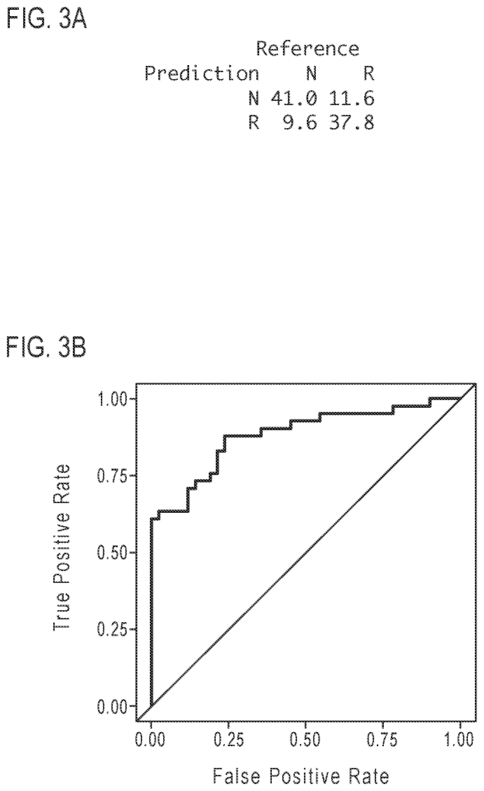

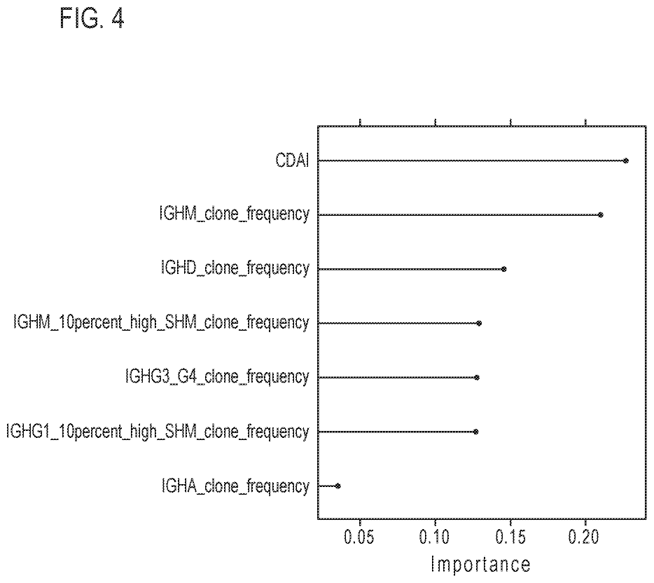

The performance results show a prediction accuracy 79%. The sensitivity and specificity were both determined to be at 78%. FIG. 3A shows the confusion matrix for the prediction of responders (R) and non-responders (N). FIG. 3B shows the ROC curve (receiver operating characteristic curve) of the final model on the dataset. The AUC-ROC (area under the ROC curve) is 0.88. FIG. 4 shows a plot of the relative importance of the features in the final prediction.

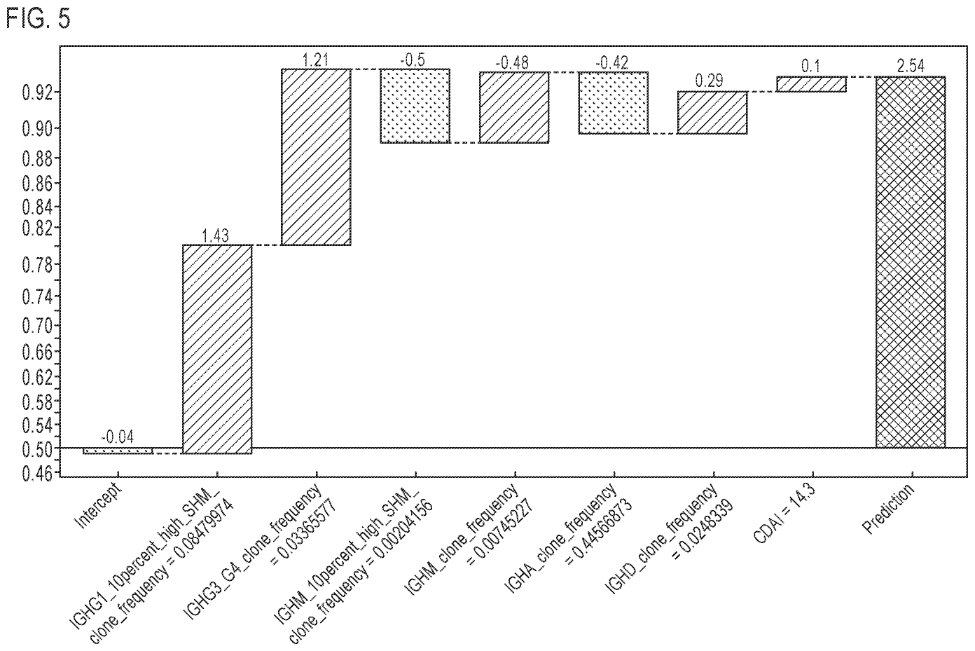

FIG. 5 shows an example of a waterfall chart illustrating the relative impact of the features on the probability of response for observation #39 (subject #39). The x-axis gives the features and the y-axis gives the log-odds. The probability of response (a continuous metric between 0 and 1, with 0 being the non-responder extreme, and 1 being the responder extreme) is the logistic function applied to the log-odds. In this example, the log-odds computed by the decision tree model for this observation is 2.54 (indicated by the black bar “Prediction”), which when converted to probability scale is 1/(1+exp(−2.54))=0.927, indicating a responder. This agrees with the truth data for subject #39, which also indicate a responder. The blue bars (positive values) show contributions of features towards calling the observation a responder and the red bars (negative values) show contributions of features towards calling the observation a non-responder. FIG. 5 shows that the biggest positive contributions to predicting this observation as a responder were the features: IGHG1 high SHIM clone frequency and IGHG3+IGHG4 clone frequency.

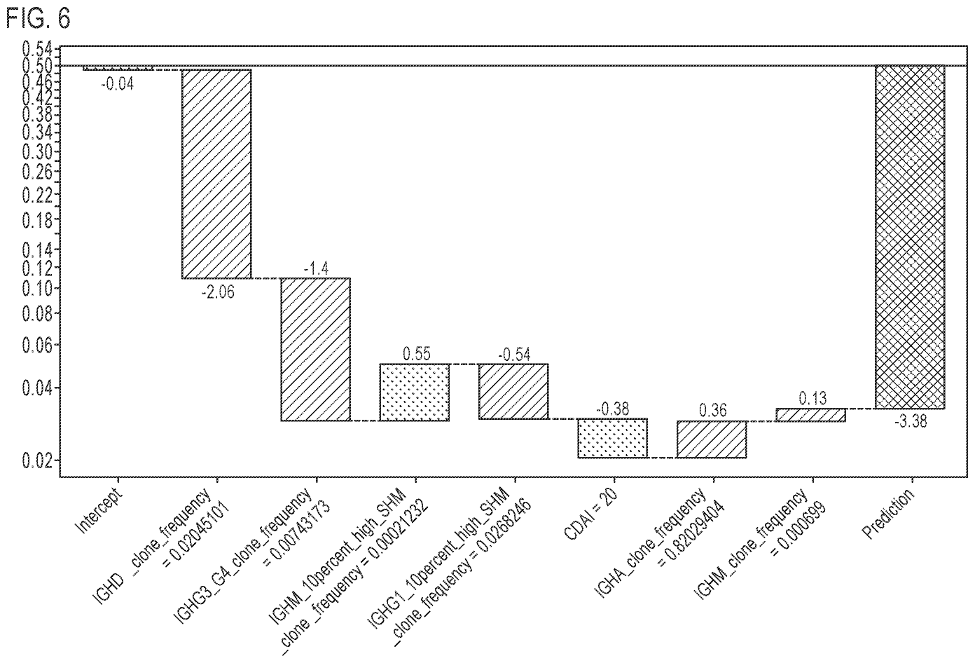

FIG. 6 shows an example of a waterfall chart illustrating the relative impact of the features on the probability of response for observation #52 (subject #52). The log-odds prediction of −3.38 is 0.033 on the probability scale, indicating a non-responder. This agrees with the truth data for subject #52, which also indicate a non-responder. In this example, the two highest contributing features are IGHD clone frequency and IGHG3+IGHG4 clone frequency. The examples of FIG. 5 and FIG. 6 show that the relative importance of features to predictions of responder or non-responder can vary for different subjects.

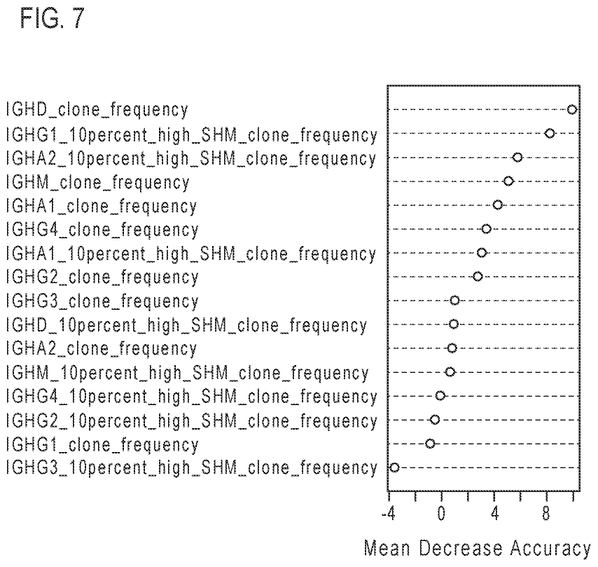

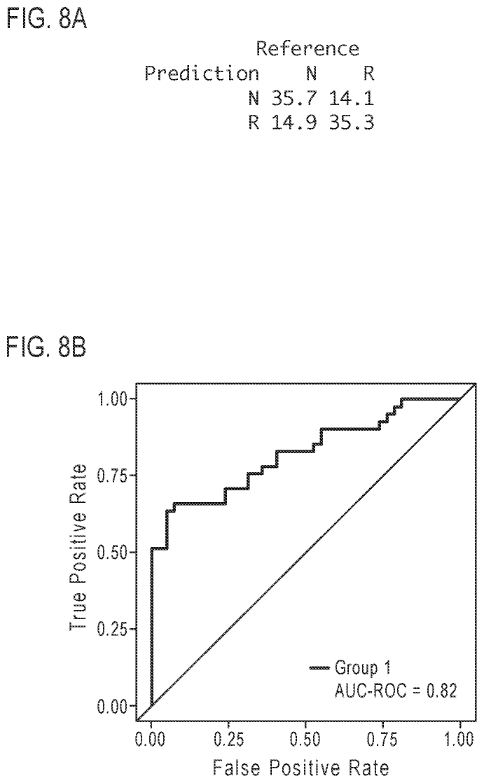

In some embodiments, five features, including IGHM clone frequency, IGHD clone frequency, IGHA clone frequency, IGHA2 highly mutated (>10%) clone frequency, IGHG1 highly mutated (>10%) clone frequency may be used. These features were selected by an optimized random forest (RF) model from the set of sixteen features shown in FIG. 7. This set of features did not include CDAI. A plurality of decision criteria was applied to these features to predict whether the subject in the is a likely responder/non-responder to treatment. In this example, the plurality of decision criteria included 100 component decision trees. As described above, each component decision tree applied at least one threshold to at least one feature to provide a corresponding output value. In this example, 100 component decision trees produced 100 output values v(i). The sum of the output values was calculated as SUM=v(1)+v(2)+ . . . v(N), where v(i) is the output value from the i-th decision tree and N=100 for this example. The sigmoid function was applied to the sum to give the prediction value, as described with respect to equation (1). The prediction value was compared to a final threshold of 0.5 to identify the subject as a likely responder or a likely non-responder to methotrexate treatment. The performance results show a prediction accuracy 71%. The sensitivity is 70% and specificity is 71%. FIG. 8A shows the confusion matrix for the prediction of responders (R) and non-responders (N). FIG. 8B shows the ROC curve of the final model on the dataset. The AUC-ROC (area under the ROC curve) is 0.82.

Example 1 is a method of predicting a clinical response to a therapy of a subject with an autoimmune disease based on B cell immune repertoire of the subject comprising: determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SEIM) level clone frequency, an IgG1 with a second somatic hypermutation (SEIM) level clone frequency; applying a plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values; summing the plurality of output values to give a summed value; applying a sigmoid transformation to the summed value to form a prediction value; and comparing the prediction value to a final threshold to identify the subject as a likely responder or a likely non-responder to an autoimmune disease therapy.

Example 2 includes the subject matter of Example 1, and further specifies that the final threshold is 0.5, and further specifies that the prediction value greater than 0.5 identifies the subject as a likely responder and the prediction value of less than or equal to 0.5 identifies the subject as a likely non-responder.

Example 3 includes the subject matter of Example 1, and further specifies that the autoimmune disease therapy comprises methotrexate.

Example 4 includes the subject matter of Example 1, and further specifies that the autoimmune disease is rheumatoid arthritis.

Example 5 includes the subject matter of Example 1, and further specifies that the plurality of features further includes a clinical disease activity index score (CDAI).

Example 6 includes the subject matter of Example 1, and further specifies that a sum of the IgG3 clone frequency and the IgG4 clone frequency provides a feature for the plurality of features.

Example 7 includes the subject matter of Example 1, and further specifies that the first somatic hypermutation (SEIM) level is greater than 10% in the IgM with the first somatic hypermutation (SEIM) level clone frequency.

Example 8 includes the subject matter of Example 1, and further specifies that the second somatic hypermutation (SEIM) level is greater than 10% in the IgG1 with the second somatic hypermutation (SEIM) level clone frequency.

Example 9 includes the subject matter of Example 1, and further specifies that the plurality of decision criteria comprises a plurality of decision trees.

Example 10 includes the subject matter of Example 9, and further specifies that at least one decision tree of the plurality of decision trees applies a first threshold to a first feature of the plurality of features followed by applying a second threshold to a second feature of the plurality of features to determine the corresponding output value.

Example 11 includes the subject matter of Example 9, and further specifies that at least one decision tree of the plurality of decision trees applies a first threshold to a first feature of the plurality of features followed by applying a second threshold to the first feature to determine the corresponding output value.

Example 12 includes the subject matter of Example 9, and further specifies that at least one decision tree of the plurality of decision trees applies a first threshold to a first feature of the plurality of features to determine the corresponding output value.

Example 13 includes the subject matter of Example 9, and further specifies that at least one decision tree of the plurality of decision trees has three predetermined output values, wherein the corresponding output value of the decision tree is selected from the three predetermined output values based on the at least one threshold applied to the at least one feature.

Example 14 includes the subject matter of Example 9, and further specifies that at least one decision tree of the plurality of decision trees has four predetermined output values, wherein the corresponding output value of the decision tree is selected from the four predetermined output values based on the at least one threshold applied to the at least one feature.

Example 15 includes the subject matter of Example 9, and further specifies that the plurality of decision trees comprises 76 decision trees.

Example 16 is system for predicting a clinical response to a therapy of a subject with an autoimmune disease based on B cell immune repertoire of the subject, including a processor and a data store communicatively connected with the processor, the processor configured to execute instructions, which, when executed by the processor, cause the system to perform a method, including: determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SEIM) level clone frequency, an IgG1 with a second somatic hypermutation (SEIM) level clone frequency; applying a plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values; summing the plurality of output values to give a summed value; applying a sigmoid transformation to the summed value to form a prediction value; and comparing the prediction value to a final threshold to identify the subject as a likely responder or a likely non-responder to an autoimmune disease therapy.

Example 17 includes the subject matter of Example 16, and further specifies that the final threshold is 0.5, and further specifies that the prediction value greater than 0.5 identifies the subject as a likely responder and the prediction value of less than or equal to 0.5 identifies the subject as a likely non-responder.

Example 18 includes the subject matter of Example 16, and further specifies that the autoimmune disease therapy comprises methotrexate.

Example 19 includes the subject matter of Example 16, and further specifies that the autoimmune disease is rheumatoid arthritis.

Example 20 includes the subject matter of Example 16, and further specifies that the plurality of features further includes a clinical disease activity index score (CDAI).

Example 21 includes the subject matter of Example 16, and further specifies that a sum of the IgG3 clone frequency and the IgG4 clone frequency provides a feature for the plurality of features.

Example 22 includes the subject matter of Example 16, and further specifies that the first somatic hypermutation (SEIM) level is greater than 10% in the IgM with the first somatic hypermutation (SEIM) level clone frequency.

Example 23 includes the subject matter of Example 16, and further specifies that the second somatic hypermutation (SEIM) level is greater than 10% in the IgG1 with the second somatic hypermutation (SEIM) level clone frequency.

Example 24 includes the subject matter of Example 16, and further specifies that the plurality of decision criteria comprises a plurality of decision trees.

Example 25 includes the subject matter of Example 24, and further specifies that at least one decision tree of the plurality of decision trees applies a first threshold to a first feature of the plurality of features followed by applying a second threshold to a second feature of the plurality of features to determine the corresponding output value.

Example 26 includes the subject matter of Example 24, and further specifies that at least one decision tree of the plurality of decision trees applies a first threshold to a first feature of the plurality of features followed by applying a second threshold to the first feature to determine the corresponding output value.

Example 27 includes the subject matter of Example 24, and further specifies that at least one decision tree of the plurality of decision trees applies a first threshold to a first feature of the plurality of features to determine the corresponding output value.

Example 28 includes the subject matter of Example 24, and further specifies that at least one decision tree of the plurality of decision trees has three predetermined output values, wherein the corresponding output value of the decision tree is selected from the three predetermined output values based on the at least one threshold applied to the at least one feature.

Example 29 includes the subject matter of Example 24, and further specifies that at least one decision tree of the plurality of decision trees has four predetermined output values, wherein the corresponding output value of the decision tree is selected from the four predetermined output values based on the at least one threshold applied to the at least one feature.

Example 30 includes the subject matter of Example 24, and further specifies that the plurality of decision trees comprises 76 decision trees.

Example 31 is a non-transitory computer-readable medium storing instructions that, when executed by a computer, cause the computer to perform a method for predicting a clinical response to a therapy of a subject with an autoimmune disease based on B cell immune repertoire of the subject, the method including: determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SEIM) level clone frequency, an IgG1 with a second somatic hypermutation (SHM) level clone frequency; applying a plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values; summing the plurality of output values to give a summed value; applying a sigmoid transformation to the summed value to form a prediction value; and comparing the prediction value to a final threshold to identify the subject as a likely responder or a likely non-responder to an autoimmune disease therapy.

Example 32 includes the subject matter of Example 31, and further specifies that the final threshold is 0.5, and further specifies that the prediction value greater than 0.5 identifies the subject as a likely responder and the prediction value of less than or equal to 0.5 identifies the subject as a likely non-responder.

Example 33 includes the subject matter of Example 31, and further specifies that the autoimmune disease therapy comprises methotrexate.

Example 34 includes the subject matter of Example 31, and further specifies that the autoimmune disease is rheumatoid arthritis.

Example 35 includes the subject matter of Example 31, and further specifies that the plurality of features further includes a clinical disease activity index score (CDAI).

Example 36 includes the subject matter of Example 31, and further specifies that a sum of the IgG3 clone frequency and the IgG4 clone frequency provides a feature for the plurality of features.

Example 37 includes the subject matter of Example 31, and further specifies that the first somatic hypermutation (SEIM) level is greater than 10% in the IgM with the first somatic hypermutation (SEIM) level clone frequency.

Example 38 includes the subject matter of Example 31, and further specifies that the second somatic hypermutation (SEIM) level is greater than 10% in the IgG1 with the second somatic hypermutation (SEIM) level clone frequency.

Example 39 includes the subject matter of Example 31, and further specifies that the plurality of decision criteria comprises a plurality of decision trees.

Example 40 includes the subject matter of Example 39, and further specifies that at least one decision tree of the plurality of decision trees applies a first threshold to a first feature of the plurality of features followed by applying a second threshold to a second feature of the plurality of features to determine the corresponding output value.

Example 41 includes the subject matter of Example 39, and further specifies that at least one decision tree of the plurality of decision trees applies a first threshold to a first feature of the plurality of features followed by applying a second threshold to the first feature to determine the corresponding output value.

Example 42 includes the subject matter of Example 39, and further specifies that at least one decision tree of the plurality of decision trees applies a first threshold to a first feature of the plurality of features to determine the corresponding output value.

Example 43 includes the subject matter of Example 39, and further specifies that at least one decision tree of the plurality of decision trees has three predetermined output values, wherein the corresponding output value of the decision tree is selected from the three predetermined output values based on the at least one threshold applied to the at least one feature.

Example 44 includes the subject matter of Example 39, and further specifies that at least one decision tree of the plurality of decision trees has four predetermined output values, wherein the corresponding output value of the decision tree is selected from the four predetermined output values based on the at least one threshold applied to the at least one feature.

Example 45 includes the subject matter of Example 39, and further specifies that the plurality of decision trees comprises 76 decision trees.

What is claimed is:

1. A method of predicting a clinical response to a therapy of a subject with an autoimmune disease based on B cell immune repertoire of the subject comprising:
determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SHM) level clone frequency, an IgG1 with a second somatic hypermutation (SEIM) level clone frequency;
applying a plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values;
summing the plurality of output values to give a summed value;
applying a sigmoid transformation to the summed value to form a prediction value; and
comparing the prediction value to a final threshold to identify the subject as a likely responder or a likely non-responder to an autoimmune disease therapy.

2. The method of claim 1, wherein the final threshold is 0.5, wherein the prediction value greater than 0.5 identifies the subject as a likely responder and the prediction value of less than or equal to 0.5 identifies the subject as a likely non-responder.

3. The method of claim 1, wherein the autoimmune disease therapy comprises methotrexate.

4. The method of claim 1, wherein the autoimmune disease is rheumatoid arthritis.

5. The method of claim 1, wherein the plurality of features further includes a clinical disease activity index score (CDAI).

6. The method of claim 1, wherein a sum of the IgG3 clone frequency and the IgG4 clone frequency provides a feature for the plurality of features.

7. The method of claim 1, wherein the first somatic hypermutation (SHM) level is greater than 10% in the IgM with the first somatic hypermutation (SHM) level clone frequency.

8. The method of claim 1, wherein the second somatic hypermutation (SHM) level is greater than 10% in the IgG1 with the second somatic hypermutation (SHM) level clone frequency.

9. The method of claim 1, wherein the plurality of decision criteria comprises a plurality of decision trees.

10. The method of claim 9, wherein at least one decision tree of the plurality of decision trees applies a first threshold to a first feature of the plurality of features followed by applying a second threshold to a second feature of the plurality of features to determine the corresponding output value.

11. The method of claim 9, wherein at least one decision tree of the plurality of decision trees applies a first threshold to a first feature of the plurality of features followed by applying a second threshold to the first feature to determine the corresponding output value.

12. The method of claim 9, wherein at least one decision tree of the plurality of decision trees applies a first threshold to a first feature of the plurality of features to determine the corresponding output value.

13. The method of claim 9, wherein at least one decision tree of the plurality of decision trees has three predetermined output values, wherein the corresponding output value of the decision tree is selected from the three predetermined output values based on the at least one threshold applied to the at least one feature.

14. The method of claim 9, wherein at least one decision tree of the plurality of decision trees has four predetermined output values, wherein the corresponding output value of the decision tree is selected from the four predetermined output values based on the at least one threshold applied to the at least one feature.

15. The method of claim 9, wherein the plurality of decision trees comprises 76 decision trees.

16. A system for predicting a clinical response to a therapy of a subject with an autoimmune disease based on B cell immune repertoire of the subject, comprising a processor and a data store communicatively connected with the processor, the processor configured to execute instructions, which, when executed by the processor, cause the system to perform a method, including:
determining a plurality of clone frequencies in a biological sample from the subject, wherein the clone frequencies include an IgM clone frequency, an IgD clone frequency, an IgG3 clone frequency, an IgG4 clone frequency and an IgA clone frequency, an IgM with a first somatic hypermutation (SHM) level clone frequency, an IgG1 with a second somatic hypermutation (SEIM) level clone frequency;
applying a plurality of decision criteria to a plurality of features including the plurality of clone frequencies, wherein each decision criterion applies at least one threshold to at least one feature of the plurality of features to provide a corresponding output value, wherein the plurality of decision criteria provides a plurality of output values;
summing the plurality of output values to give a summed value;
applying a sigmoid transformation to the summed value to form a prediction value; and
comparing the prediction value to a final threshold to identify the subject as a likely responder or a likely non-responder to an autoimmune disease therapy.

17. The system of claim 16, wherein the final threshold is 0.5, wherein the prediction value greater than 0.5 identifies the subject as a likely responder and the prediction value of less than or equal to 0.5 identifies the subject as a likely non-responder.

18. The system of claim 16, wherein the autoimmune disease therapy comprises methotrexate.

19. The system of claim 16, wherein the autoimmune disease is rheumatoid arthritis.

20. The system of claim 16, wherein the plurality of features further includes a clinical disease activity index score (CDAI).
